(19) United States
(12) Patent Application Publication
(10) Pub. No.: US 2004/0019092 A1
(43) Pub. Date: Jan. 29, 2004

(54) Non-peptide somatostatin receptor ligands

(76) Inventors: Daniel Berney, Lausanne (CH); Robin Breckenridge, Hagental-le-bas (CH); Peter Neumann, Berne (CH); Gideon Shapiro, Gainesville, FL (US); Max Peter Seiler, Riehen (CH); J. Troxler Thomas, Wahlen (CH)

Correspondence Address: KING & SPALDING, 191 PEACHTREE STREET, N.E., ATLANTA, GA, 30303-1763, US

(21) Appl. No.: 10/295,079

(22) Filed: Nov. 14, 2002

Related U.S. Application Data
(60) Provisional application No. 60/333,239, filed on Nov. 14, 2001.

(51) Int. Cl. C07D 403/02; A61K 31/4178

(52) U.S. Cl. 514/389; 548/312.1

(57) ABSTRACT

The present invention provides compounds of formula (I), wherein X, Y, R1, R2, R3, and R4 are as defined in the description, and the preparation therof. The compounds of the formula bind to somatostatin receptiors and are useful as pharmaceuticals.

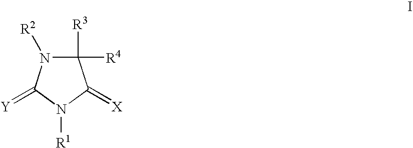

This application claims priority to U.S. Provisional Application No. 60/333,239, filed on Nov. 14, 2001.

The present invention provides novel hydantoin derivatives, their preparation, their use as pharmaceuticals and pharmaceutical compositions containing them.

The invention provides compounds of formula I.

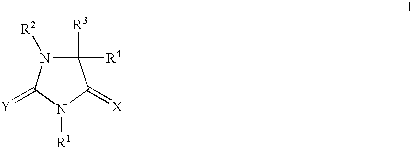

wherein

X and Y independently are O or H, H;

R¹ is a group of formula:

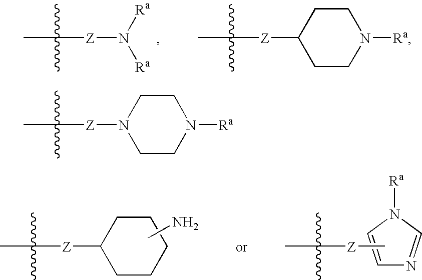

wherein

R^{a} independently are hydrogen, C₁₋₄ alkyl or a CH₃COO—CH(CH₃)—OCO— group; and

Z is a saturated or unsaturated aliphatic C₂₋₆ hydrocarbonic chain which is (a) optionally interrupted by —O— or —S— and (b) optionally substituted by C₁₋₄ alkyl or C₁₋₄ alkoxy groups;

R² is a group of formula —SO₂-Aᵣ of —CH₂—Ar

wherein

Ar is phenyl or naphthyl optionally mono- or di-substituted by hydroxy, halogen, C₁₋₄ alkyl, C₁₋₄ alkoxy, cyano, trifluromethyl, aminomethyl, dimethylamincarbonyl, benximidazolyloxy or morpholinocarbonyl, or by a group of formula:

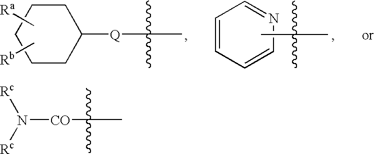

wherein

Q is CH₂, O, S or CO,

R^{b} independently are hydrogen, C₁₋₄ alkyl, C₁₋₄ alkoxy, amino, halogen, hydroxy, a NH₂—(CH₂)₄—CH(NH₂)—COO— group or form together a methylenedioxy, and

R^{c} independently is hydrogen or C₁₋₄ alkyl.

R³ is hydrogen or C₁₋₄ alkyl; and

R⁴ is a group of formula:

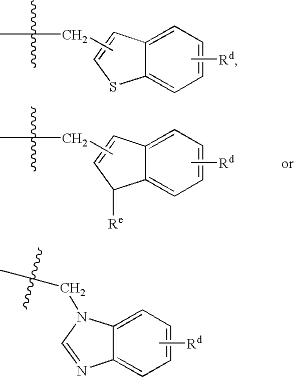

wherein

R^{d} is hydrogen, halogen, C₁₋₄ alkyl or C₁₋₄ alkoxy, and

R^{e} is hydrogen, C₁₋₄ alkyl or benzyl, in free base or acid addition salt form.

X and Y are preferably O;

R¹ preferably is -Z-NH₂, wherein Z is preferably an alkylene chain;

R² preferably is —SO₂—Ar, wherein Ar is preferably an optionally substituted phenyl;

R³ preferably is H; and/or

R⁴ is preferably an optional substituted 3-indolyl.

An alkyl or alkoxy group as defined above preferably has one or two carbon atoms and more preferably is methyl or methoxy.

Depending on the nature of the substituents defined above, one or more asymmetric carbons may be present in the molecule. All optical isomers and their mixtures including the racemic mixtures are part of the present invention.

The compounds of formula I may be prepared over a process that includes the steps of (a) reacting a compound of formula II

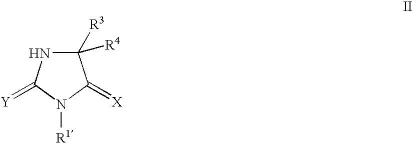

wherein X, Y, R³ and R² are as defined above and R^{1′} is R¹ as defined above or a protected form of R¹, with a compound of formula III.

R^{2′}-Hal III

wherein R^{2′} is R² as defined above or a protected form of R² and Hal is chlorine, bromine or iodine; and

(b) deprotecting the resulting product and recovering the thus obtained compound of formula I in free base or acid addition salt form.

A protected amino group of R^{1′} is for example an N-butyloxycarbonyl (Boc)- or an N₃— residue.

When in formula III, R^{2′} is a group of formula —SO₂—Ar, Hal is preferably chlorine.

The condensation of the compound of formula II with the compound of formula III and the subsequent deprotection can be effected according to known methods, for example as described in Example 3.

Working up the reaction mixtures obtained and purification of the compounds of formula I may also be carried out in accordance with known methods.

Acid addition salts may be produced from the free bases in known manner, and vice versa.

The starting compounds of formula II are known or may be produced by known methods. For example compounds of formula II wherein X and Y are O may be produced in accordance with the following reaction scheme, for example as described in Example 1:

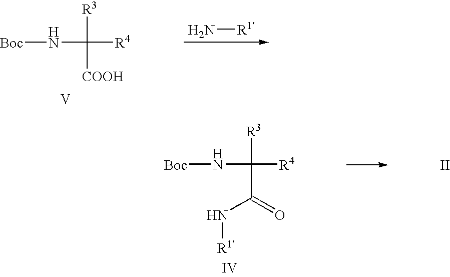

The starting compounds of formulae III and V are known or may be produced by known processes.

The compounds of formula I and their physiologically acceptable acid addition salts, hereinafter referred to as compounds of the invention, have interesting pharmacological properties when tested in vitro using SRIF receptor expressing cell cultures and in animals, and may therefore be used as pharmaceuticals.

In particular the compounds of the invention bind to somatostatin receptors. More particularly they are selective agonists at Somatostatin sst₂ receptors, as determined in radioligand binding and second messenger studies (see for example K. Kaupmann et al., FEBS LETTERS 1993, 331, 53-50).

The compounds of the invention are therefore indicated for use in anxiety, depression, schizophrenia, neurodegenerative diseases such as dementia, epilepsy, endrocrinological disorders associated with an excess of hormone release such as: growth hormone (GH) glucagon or insulin secretion, gastro-intestinal disorders, for the treatment of tumors and for vascular disorders and immunological diseases.

The usefulness of the compounds of the invention in these indications is confirmed in a range of standard tests as indicated below.

At doses of about 0.3 to 3 mg/kg p.o., the compounds of the invention increase exploratory behavior of mice in the open half of the half enclosed platform, a model which is predictable for anxiolytic activity (Psychopharmacology, 1986, 89, 31-37).

In the same half enclosed platform model, the compounds of the invention at the above indicated doses increase vigilance and exploratory components of behavior of the mice. The compounds are therefore indicated for the treatment of depression, schizophrenia and dementia, in particular of senile dementia of the Alzheimer type (SDAT). In addition, there is circumstantial clinical evidence for various types of dementias to be associated with reduced somatostatin levels (see for example J. Epelbaum et al., Clinical Reviews in Neurobiology, 1994, 8, 25-44).

At doses of about 0.3 to 3 mg/kg p.o., the compounds of the invention inhibit epileptic seizure in electrically and chemically induced episodes in rats (A. Vezzani et al., Neuropharmacol., 1991, 30, 345-352).

Furthermore the compounds of the invention inhibit GH release in cultured pituitary cells in vitro and depress serum GH and insulin levels in the rat. The test is carried out using male rats. The test substance is administered at varying, logarithmically staggered doses employing at least 5 rats per dose. One hour after subcutaneous (s.c.) administration of the test substance blood is taken. The determination of the blood serum GH and insulin levels is measured by radio-immunoassay. The compounds of the invention are active in this test when administered at a dosage in the range of from 0.1 to 1 mg/kg s.c.

The inhibitory effect of the compounds on GH release may also be examined after oral application to male rats with oestradiol implants. This test is carried out as follows.

A loop (length 50 mmØ3 mm) of silastic with 50 mg of oestradiol is implanted under the dorsal skin of anaesthetized male OFA rats that have a weight of ca. 300 g. At various times (1 to 6 months later), these animals, in a fasted state, are used repeatedly for tests. The test substances are active in this test at doses from 0.1 to 5 mg/kg, when GH level in the blood serum is determined by radio-immunoassay 1 and 2 hours after oral administration.

The compounds of the invention are accordingly indicated for use in the treatment of disorders with an etiology comprising or associated with excess GH-secretion, e.g., in the treatment of acromegaly as well as in the treatment of diabetes mellitus, especially complications thereof, e.g., angiopathy, proliferative retinopathy, dawn phenomenon and nephropathy.

The compounds of the invention also inhibit gastric and exocrine and endocrine pancreatic secretion and the release of various peptides of the gastrointestinal tract, as indicated in standard tests using e.g. rats with gastric and pancreatic fistulae.

The compounds are thus additionally indicated for use in the treatment of gastro-intestinal disorders, for example in the treatment of peptic ulcers, disturbances of GI motility, enterocutaneous and pancreaticocutaneous fistula, irritable bowel syndrome, dumping syndrome, watery diarrhea syndrome, acute pancreatitis and gastro-intestinal hormone secreting tumors (e.g., vipomas, glucagonomas, insulinomas, carcinoids and the like) as well as gastro-intestinal bleeding (see for example Th. O'Dorisio et al., Advances Endocrinol. Metab., 1990, 1, 175-230).

The compounds of the invention are also effective in the treatment of various kinds of tumors, particularly of SSTR-2 receptor bearing tumors, as indicated in proliferation tests with various cancer cell lines and in tumor growth experiments in nude mice with hormone dependent tumors (see for example G. Weckbecker et al., Cancer Research 1994, 54, 6334-6337). Thus the compounds can be used in the treatment of, for example, cancers of the breast, the prostate, the colon, the pancreas, the brain and the lung (small cell lung cancer).

For the above-mentioned indications, the appropriate dosage will of course vary depending upon, for example, the compound employed, the host, the mode of administration and the nature and severity of the condition being treated. However, in general, satisfactory results in animals are indicated to be obtained at a daily dosage of from 0.1 to about 50, preferably from about 0.5 to about 20 mg/kg animal body weight. In larger mammals, for example humans, an indicated daily dosage is in the range from about 1 to about 100, preferably from about 5 to about 50 mg of an agent of the invention conveniently administered, for example, in divided doses up to four times a day or in sustained release form.

The compounds of the invention may be administered in free form or in pharmaceutically acceptable salt form or complexes. Such salts and complexes may be prepared in conventional manner and exhibit the same order of activity as the free compounds.

The present invention also provides a pharmaceutical composition comprising a compound of the invention in free base form or in pharmaceutically acceptable acid addition salt form in association with a pharmaceutically acceptable diluent or carrier. Such compositions may be formulated in conventional manner. The compounds may be administered by any conventional route, for example parenterally e.g. in form of injectable solutions or suspensions, enterally, preferably orally, e.g. in the form of tablets or capsules or in a nasal or a suppository form.

Moreover the present invention provides the use of the compounds of the invention for the manufacture of a medicament for the treatment of any condition mentioned above.

In still a further aspect the invention provides a method for the treatment of any condition mentioned above, in a subject in need of such treatment, which comprises administering to such subject a therapeutically effective amount of a compound of the invention.

Compounds of the present invention having a chiral center may exist in and be isolated in optically active and racemic forms. Some compounds may exhibit polymorphism. The present invention encompasses racemic, optically-active, polymorphic, or stereoisomeric form, or mixtures thereof, of a compound of the invention, which possess the useful properties described herein. The optically active forms can be prepared by, for example, resolution of the racemic form by recrystallization techniques, by synthesis from optically-active starting materials, by chiral synthesis, or by chromatographic separation using a chiral stationary phase or by enzymatic resolution.

### BRIEF DESCRIPTION OF THE FIGURES

FIG. 1 is an illustration of non-limiting examples of hydantoins of the present invention.

FIG. 2 is a line graph depicting the effect of compound 45 on growth hormone (GH) plasma levels in the Rhesus monkey after subcutaneous administration in two hour intervals. Data in percent of basal values at time zero.

FIG. 3 is a bar graph of the dose-dependent effects of subcutaneous administration of compound 14 on cerebral cortex somatostatin (SRIF) binding sites in Wistar rats where the bars represent specific binding that remain following treatment compared to control rats receiving saline. Similarly, the effects on binding of [¹²⁵I]-labeled Tyr³ analogue of Octreotride™ and [¹²⁵I]SRIF-28 (somatostatin-28) are illustrated.

FIG. 4 is a bar graph of the dose-dependent effects of subcutaneous administration of compound 14 on hippocampal SRIF binding sites in Wistar rats where the bars represent specific binding that remain following treatment compared to control rats receiving saline. Similarly, the effects on binding of [¹²⁵1]-labeled Tyr³ analogue of Octreotride™ and [¹²⁵I]SRIF-28 are illustrated.

FIG. 5 is a line graph of blood concentrations in three male Wistar rats of compound 42 after intravenous and oral administration.

FIG. 6 is a line graph of blood concentrations in three male Wistar rats of radiolabeled and non-radiolabeled compound 42 after an intravenous bolus of 1 mg/kg.

FIG. 7 is a radiochromatogram of blood extracts: pools from 6 rats, different times after a single 1 mg/kg intravenous dose of [¹⁴C]-42, and blank rat blood spiked with ˜100 μ/mL of [¹⁴C]-42.

FIG. 8 is a radiochromatogram of selected rat urine samples, collected 0-48 hours after a single oral (10 mg/kg) and intravenous (1 mg/kg) dose of [¹⁴C]-42.

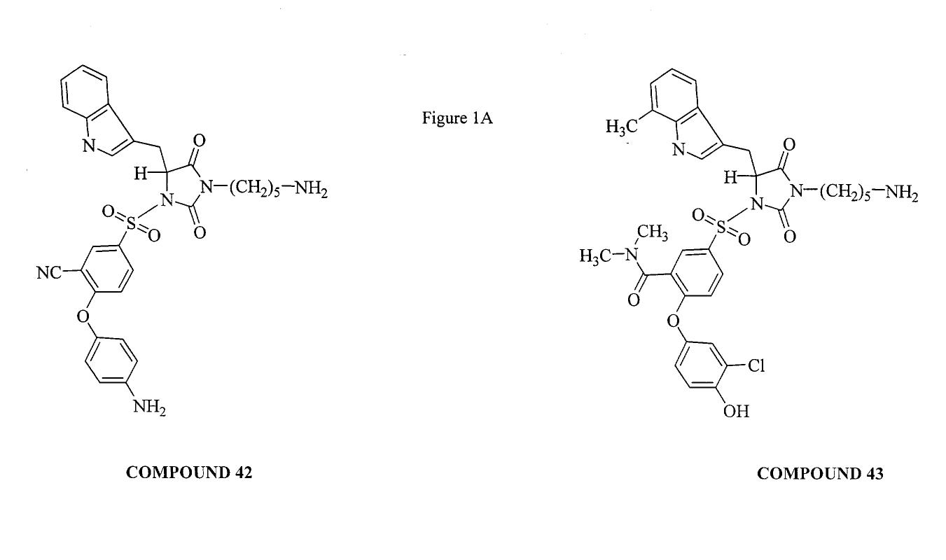

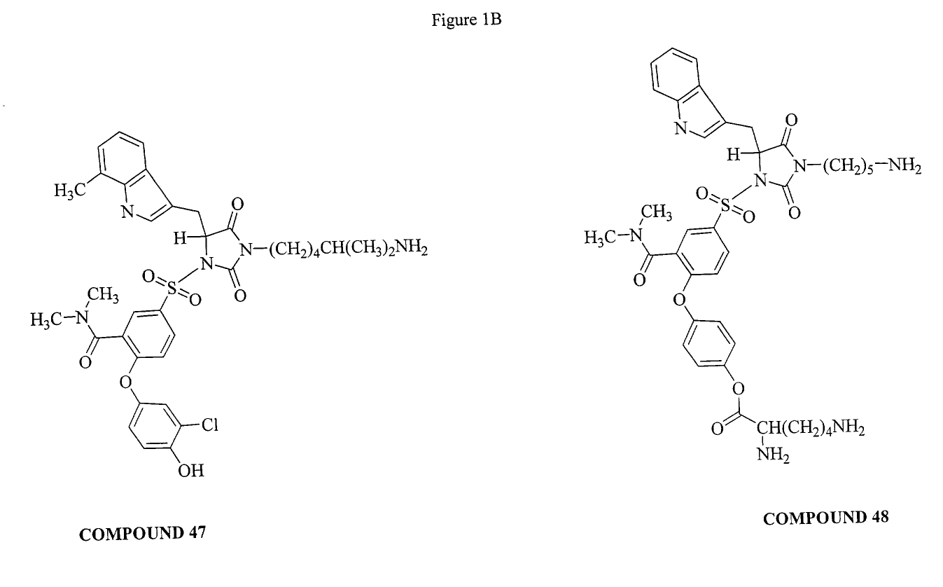

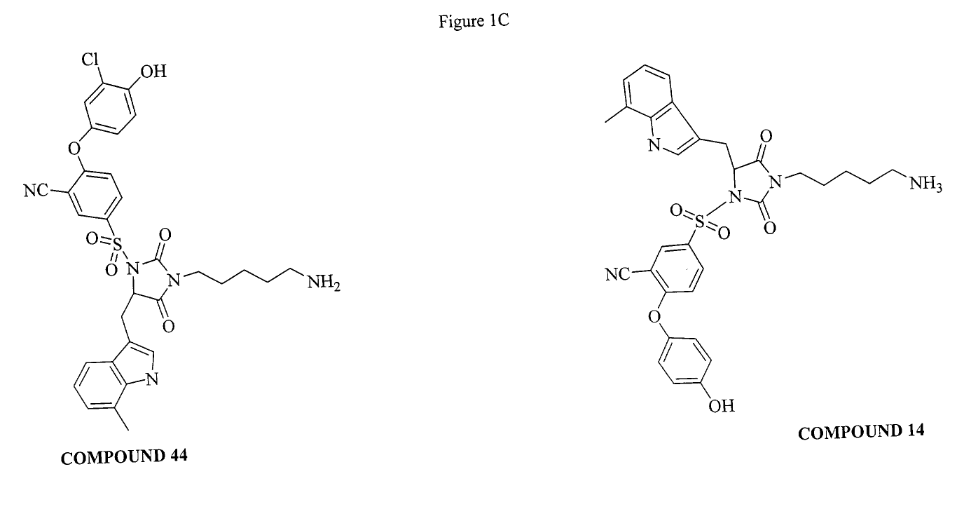

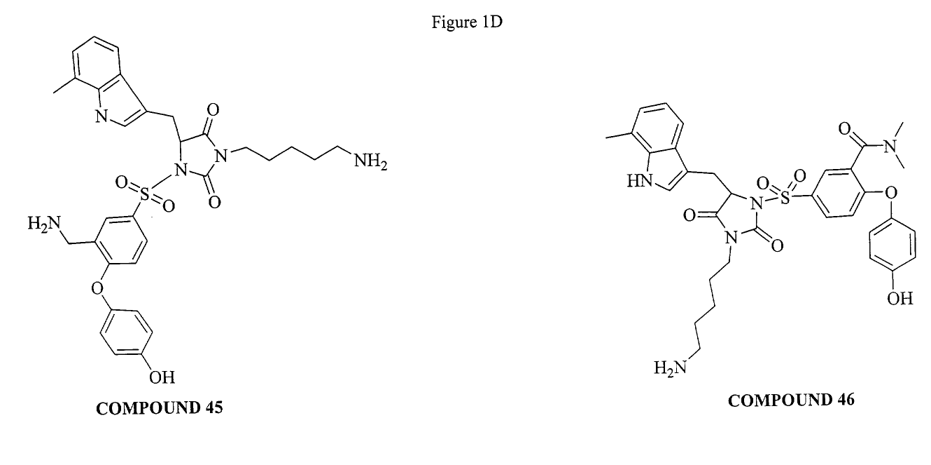

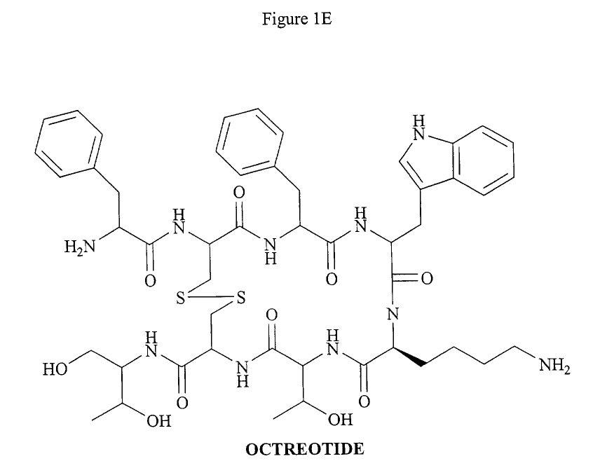

The following examples illustrate the invention. The temperatures are given in degrees Celsius and are uncorrected.

### EXAMPLE 1

N-a-t-butyloxycarbonyl-d,l-tryptophan-[5-amino-(N-t-butyloxycarbonyl)-n-pentanyl]amide

To a stirred solution of mono-N-Boc-1,5-pentanediamine (1.27 g, 6.3 mmol) and d,l-tryptophan (2.12 g, 7.0 mmol) in 30 mL THF is added dicyclohexylcarbodiimide (DCC) (1.54 g, 7.5 mmol) at room temperature. After one hour the mixture is filtered to remove the precipitated dicyclohexylurea and concentrated in vacuo. Ether is added, the mixture is filtered and then cooled, whereupon the product crystallizes out of solution. Filtration yields the product 1 as a light brown powder; mp. 97-98°.

### EXAMPLE 2

3-[5′-amino-(N-t-butyloxycarbonyl)-n-pentanyl]-5-[(indol-3-yl)-methyl]-imidazolidine-2,4-dione (2)

Compound 1 (2.93 g, 6.0 mmol) is dissolved in 50 mL THF and heated under reflux with tetrabutylammonium fluoride trihydrate (5.68 g, 18 mmol). After 24 hours the mixture is concentrated in vacuo. The residue is dissolved in ethyl acetate, extracted with brine, dried (sodium sulfate) and concentrated to a viscous brown oil. Medium pressure liquid chromatography (MPLC) (138 g SiO₂; ethyl acetate:hexane 2:1) gives the product 2 as a light yellow oil which crystallizes upon standing. An analytical sample is prepared by recrystallization from ethyl acetate-hexane; mp. 136-137°.

### EXAMPLE 3

(+/−)-1-(2′,5′-dichloro-1′-benzenesulfonyl)-3-(5′-amino-n-pentanyl)-5-[(indol-3-yl)-methyl)]-imidazolidine-2,4-dione (3)

Sodium hexamethyldisilazide (1.1 mmol, 1.1 mL 1M solution in THF) is added to a stirred solution of 3-[5′-amino-(N-t-butyloxycarbonyl)-n-pentanyl]-5-[(indol-3-yl)methyl]-imidazolidine-2,4-dione (2, 415 mg, 1.0 mmol) in 5 mL dry tetrahydrofuran (THF) at 40° under argon. After 30 minutes, 2,5-dichlorobenzenesulfonyl chloride (270 mg, 1.1 mmol) is added and the solution is allowed to stir overnight at room temperature. Saturated ammonium chloride solution is added and the mixture concentrated on a rotary evaporator. The mixture is then dissolved in ethyl acetate, extracted with brine, dried (sodium sulfate) and concentrated to a viscous oil This crude product 3 is purified by medium pressure liquid chromatography (MPLC) over silica gel (59 g SiO₂, 0.015-0.04 mm; ethylacetate hexane 2:1) to give a colorless viscous oil.

The so obtained product (530 mg, 0.85 mmol) is dissolved in 6 mL of dichloromethane and iodotrimethylsilane (240 mg, 2.0 mmol) is added. After stirring for 10 minutes at room temperature, potassium bicarbonate (4 mL, 2N solution) is added and the resulting solution stirred for 15 minutes. The organic phase is separated, dried (sodium sulfate) and concentrated to give the crude free base. This base is dissolved in 4 mL ethanol and ethereal HCl solution (1 mL, ca. 1N solution) is added. The solution is cooled and ether added whereupon the hydrochloride salt crystallizes out of solution. Filtration provides the product in hydrochloride salt form; mp. 157-159°.

The compounds of formula I wherein R¹, R², R³ and R⁴ are as defined in the following Table 1 and X and Y are both 0 as well as the compounds of formula I wherein R¹, R², R³ and R⁴ are defined in the following Table 2, X is H, H and Y is O, are prepared in analogous manner to Examples 1-3.

TABLE 1

| COMPOUND | R¹ | R² | R³ | R⁴ |
|---|---|---|---|---|
| 4 | —(CH₂)₅NH₂ | —SO₂-p-toluyl | H | —CH₂-3-indolyl |
| 5 | —(CH₂)₅NH₂ | —SO₂-3,4-(CH₃)₂-Ph | H | —CH₂-3-indolyl |
| 6 | —(CH₂)₅NH₂ | —SO₂-m-CH₃-Ph | H | —CH₂-3-indolyl |
| 7 | —(CH₂)₅NH₂ | —SO₂-o-OMe-Ph | H | —CH₂-3-indolyl |
| 8 | —CH₂—CH═CH—(CH₂)₂— | —SO₂-3,4-(OCH₃)₂-Ph | H | —CH₂-3-indolyl |
| | NH₂ (cis) | | | |
| 9 | —(CH₂)₂—O—(CH₂)₂— | —SO₂-3,4-(OCH₃)₂-Ph | H | —CH₂-3-indolyl |
| | NH₂ | | | |
| 10 | —(CH₂)₄—NH₂ | —SO₂-3,4-(OCH₃)₂-Ph | H | —CH₂-3-indolyl |
| 11 | —(CH₂)₆—NH₂ | —SO₂-3,4-(OCH₃)₂-Ph | H | —CH₂-3-indolyl |
| 12 | —(CH₂)₅—NH₂ | —SO₂-3-[(Me)₂NCO]-4-(5- | H | —CH₂-3-indolyl |
| | | benzimidazolyl-O)-Ph | | |
| 13 | —(CH₂)₅—NH₂ | —SO₂-p-(p-NH₂-Ph-O)-Ph | H | —CH₂-3-indolyl |
| 14 | —(CH₂)₅—NH₂ | —SO₂-3-CN-4-(p-OH-Ph-O)-Ph | H | —CH₂-3-(7-CH₃- |
| | | | | indolyl) |
| 15 | —(CH₂)₅—NH₂ | —SO₂-3-NH₂CH₂-4-(p-OH-Ph- | H | —CH₂-3-(7-CH₃- |
| | | O)-Ph | | indolyl) |
| 16 | —(CH₂)₅—NH₂ | —SO₂-p-(p-OH-Ph-CO)-Ph | H | —CH₂-3-indolyl |
| 17 | —(CH₂)₅—NH₂ | —SO₂-p-(p-OH-Ph-CH₂)-Ph | H | —CH₂-3-indolyl |
| 18 | —(CH₂)₅—NH₂ | —SO₂-p-(p-OH-Ph-O)-Ph | H | —CH₂-3-indolyl |
| 19 | —(CH₂)₅—NH₂ | —SO₂-p-(-OH-Ph-S)-Ph | H | —CH₂-3-indolyl |
| 20 | —(CH₂)₅—NH₂ | —SO₂-p-(4-NH₂-2-pyridyl-O)-Ph | H | —CH₂-3-indolyl |
| 21 | —(CH₂)₅—NH₂ | —SO₂-3-(morpholino-CO)-4-(p- | H | —CH₂-3-indolyl |
| | | Cl-Ph-O)-Ph | | |
| 22 | 4-piperidinyl-(CH₂)2- | —SO₂-3-[(Me)₂NCO]-4-(p-OH- | H | —CH₂-3-indolyl |
| | | Ph-O)-Ph | | |
| 23 | —(CH₂)₅—NH₂ | —SO₂-3-[(Me)₂NCO]-4-(3-Cl-4- | H | —CH₂-3-indolyl |
| | | OH-Ph-O)-Ph | | |
| 24 | 4-Me-1-piperazinyl- | —SO₂-3-[(Me)₂NCO]-4-(p-OMe- | H | —CH₂-3-indolyl |
| | (CH₂)₂— | Ph-O)-Ph | | |
| 25 | —(CH₂)₅—NH₂ | —SO₂-3-[(Me)₂NCO]-4-(NH₂— | H | —CH₂-3-indolyl |
| | | (CH₂)₄—CH(NH₂)—COO-Ph-O]- | | |
| | | Ph | | |
| 26 | [MeCOO—CH(Me)- | —SO₂-3-[(Me)₂NCO]-4-(p-OH | H | —CH₂-3-indolyl |
| | OCO—NH]—(CH₂)₅— | Ph-O)-Ph- | | |
| 27 | —(CH₂)₅—NH₂ | —SO₂-3-[(Me)₂NCO]-4-(3-Cl-4- | H | —CH₂-3-(7-Cl- |
| | | OH-Ph-O)-Ph | | indolyl) |
| 28 | —CH₂-(p-trans-NH₂- | —SO₂-3-[(Me)₂NCO]-4-(p-OH- | H | —CH₂-3-indolyl |
| | cyclohexyl) | Ph-O)-Ph | | |
| 29 | —(CH₂)₅—N(Me)₂ | —SO₂-3-[(Me)₂NCO]-4-(3-Cl-4- | H | —CH₂-3-indolyl |
| | | OH-Ph-O)-Ph | | |
| 30 | —(CH₂)₃-(1-imidazolyl) | —SO₂-3-[(Me)₂NCO]-4-(3-Cl-4- | H | —CH₂-3-indolyl |
| | | OH-Ph-O)-Ph | | |
| 31 | —(CH₂)₅—NH₂ | —SO₂-3-[(Me)₂NCO]-4-(3-Cl-4- | H | —CH₂-3-(7-Me- |
| | | OH-Ph-O)-Ph | | indolyl) |
| 32 | —(CH₂)₅—NH₂ | —SO₂-3-[(Me)₂NCO]-4-(3-Cl-4- | H | —CH₂-3-(5-Me- |
| | | OH-Ph-O)-Ph | | indolyl) |
| 33 | —(CH₂)₄—CH(Me)₂—NH₂ | —SO₂-3-[(Me)₂NCO]-4-(3-Cl-4- | H | —CH₂-3-(7-Me- |
| | | OH-Ph-O)-Ph | | indolyl) |
| 34 | —(CH₂)₃-(1-Me-4- | —SO₂-3-[(Me)₂NCO]-4-(3-Cl-4- | H | —CH₂-3-indolyl |
| | imidazolyl) | OH-Ph-O)-Ph | | |
| 35 | —(CH₂)₅—NH₂ | —SO₂-3-CH(CH₃)₂-4-(3-Cl-4- | H | —CH₂-3-(7-Me- |
| | | OH-Ph-O)-Ph | | indolyl) |
| 36 | —(CH₂)₄—C(CH₃)₂—NH₂ | —SO₂-3-[(Me)₂NCO]-4-(p-F-Ph- | H | —CH₂-3-(7-Me- |
| | | O)-Ph | | indolyl) |
| 37 | —(CH₂)₄—C(CH₃)₂—NH₂ | —CH₂-3-[(Me)₂NCO]-4-(3-Cl-4- | H | —CH₂-3-(7-Me- |
| | | MeO-Ph-O)-Ph | | indolyl) |
| 38 | —(CH₂)₄—C(CH₃)₂—NH₂ | —SO₂-3-[(Me)₂NCO]-4-(3,4- | H | —CH₂-3-(7-Me- |
| | | dichloro-Ph-O)-Ph | | indolyl) |
| 39 | —(CH₂)₄—C(CH₃)₂—NH₂ | —SO₂-3-[(Me)₂NCO]-4-(3-Cl-4- | H | —CH₂-3- |
| | | OH-Ph-O)-Ph | | benzo[b]thienyl |
| 40 | —(CH₂)₄—C(CH₃)₂—NH₂ | —SO₂-3-[(Me)₂NCO]-4-(3-Cl-4- | H | —CH₂-1-(4-Me- |
| | | MeO-Ph-O)-Ph | | benzimidazolyl) |
| 41 | —(CH₂)₄—C(CH₃)₂—NH₂ | —SO₂-3-[(Me)₂NCO]-4-[(3,4- | H | —CH₂-3-(7-Me- |
| | | methylenedioxy-Ph-O)-Ph | | indolyl) |
| 42 | —(CH₂)₅—NH₂ | —SO₂-3-(CN)-4-(p-NH₂-Ph-O)- | H | —CH₂-3-indolyl |
| | | Ph | | |
| 43 | —(CH₂)₅—NH₂ | —SO₂-3-[(Me)₂NCO]-4-(3-Cl-4- | H | —CH₂-3-(7-Me- |
| | | OH-Ph-O)-Ph | | indolyl) |
| 44 | —(CH₂)₅—NH₂ | —SO₂-3-(CN)-4-(3-Cl-4-OH-Ph- | H | —CH₂-3-(7-Me- |
| | | O)-Ph | | indolyl) |
| 45 | —(CH₂)₅—NH₂ | —SO₂-3-(CH₂NH₂)-4-(p-OH-Ph- | H | —CH₂-3-(7-Me- |
| | | O)-Ph | | indolyl) |
| 46 | —(CH₂)₅—NH₂ | —SO₂-3-[(Me)₂NCO]-4-(p-OH- | H | —CH₂-3-(7-Me- |
| | | Ph-O)-Ph- | | indolyl) |
| 47 | —(CH₂)₄—C(CH₃)₂—NH₂ | —SO₂-3-[(Me)₂NCO]-4-(3-Cl-4- | H | —CH₂-3-(7-Me- |
| | | OH-Ph-O)-Ph | | indolyl) |
| 48 | —(CH₂)₅—NH₂ | —SO₂-3-[(Me)₂NCO]-4-[3-Cl-4- | H | —CH₂-3-indolyl |
| | | (OCOCH(NH₂)(CH₂)₄NH₂)-Ph- | | |
| | | O]-Ph | | |

Me = methyl; Ph = phenyl

TABLE 2

| Cmpd. | R¹ | R² | R³ | R⁴ |
|---|---|---|---|---|
| 49 | —CH₂—CH═CH—(CH₂)₂—NH₂ | —CH₂-Ph | H | —CH₂-3- |
| | | | | indolinyl |
| 50 | —CH₂—CH═CH—(CH₂)₂—NH₂ | —CH₂-Ph | H | —CH₂-3-indolyl |
| 51 | —(CH₂)₅—NH₂ | —CH₂-Ph | H | —CH₂-3-indolyl |
| 52 | —CH₂-p-(aminomethyl)-Ph | —CH₂-Ph | H | —CH₂-3-indolyl |
| 53 | —(CH₂)₅—NH₂ | —SO₂-3,4-(OMe)₂-Ph | H | —CH₂-3-indolyl |
| 54 | —(CH₂)₅—NH₂ | —SO₂-p-(p-OH-Ph-O)-Ph | H | —CH₂-3-indolyl |

Me = methyl; Ph = phenyl

### EXAMPLE 4

Effects of Hydantoins on GH Plasma Levels in Rats

Five somatostatin (SRIF) receptor subtypes have been characterized (sst₁-sst₅). The natural ligands, somatostatin 14 (SRIF 14) and somatostatin 28 (SRIF 28), bind to all 5 receptors with high nanomolar/subnanomolar affinity, whereas the clinically used octapeptides Octreotide (Sandostatin®), BIM23014 and RC160, bind with preference to the sst₂ and to a lesser extent to sst₂ subtype (Bruns C. et al. “Molecular phamacology of somatostatin-receptor subtypes” Mol. Cell Biol Aspects Gastr Neur Tumor Dis 1994, 733, 138-146).

The in vitro binding affinities for rat cortex SRIF receptors and the human sst₂ receptor sub-type was assessed. Further the in vitro inhibition of the GHRH induced GH secretion in primary cultures of rat pituitary cells as well as the in vivo inhibition of GH secretion in the rat after systemic or enteral application was studied. Several compounds exhibited inhibiting activities in the nanomolar range, comparable to the natural ligand somatostatin (SRIF). Therefore, the in vivo activities of the compounds in rats with respect to inhibition of growth hormone release was also assessed. Most compounds selected were tested by enteral—intraduodenal (i.d.) application. For determination of the absolute activity and for the calculation of an enteral bioavailability, single subcutaneous (s.c.) application of intravenous (i.v.) infusion was used. When applied by infusion, a number of compounds with reasonable parental activities were found.

Method

Animals, anesthesia: Male rats of a Sprague Dawley strain (Ico:OFA-SD, Iffa-Credo, F-Lyon) of 200-300 g body weight were used. They were kept under standardized conditions and were anesthetized with pentobarbital-sodium (Siegfried, CH Zofingen), 60 mg/kg i.p. for subcutaneous and intraduodenal applications. Animals designed for an infusion experiment were anesthetized with urethane (Siegfried, CH Zofingen), 1.2 g/kg i.p.

Compounds: The compounds were dissolved, optionally with the aid of one or more of the following adjuncts:

Tween 80: up to 10%

Di-methyl-sulfoxide (DMSO): up to 10%

N-methyl-pyrrolicone (NMP): up to 5%

Hydrochloric acid (HCl): molar proportion

Sodium Carbonate (Na₂CO₃): molar proportion

Stock solutions of the compounds were prepared in water with the addition of the necessary adjunct(s). Further dilutions were prepared allowing 5 mL/kg for subcutaneous application, 2 mL for intraduodenal (i.d.) application or 4.5 mL/kg for intravenous infusion. The solutions for the intravenous application/infusion were made isotonic by addition of glucose to a final concentration of 5%.

Applications were made, or, in the case of infusion, started, approximately 10 minutes after induction of anesthesia. No further treatment followed subcutaneous or intraduodenal application. Ten, twenty-five and fifty-five minutes after start of the intravenous infusion the GH secretion was stimulated with repeated intravenous injections of the potent GHRH analog, D-Ala₂ GHRH₁₋₂₉NH₂, 1 μg/kg.

One hour after subcutaneous or intraduodenal application of the compounds, blood was collected from the trunk after decapitation. In the case of application by infusion, 5 minutes after each application of GHRH, a blood sample of 0.8 mL was taken from a V. jugularis and mixed with EDTA (Eppendorf tubes TOM14, Milian, CH Geneva). The blood samples were kept on ice and spun in a cooled centrifuge. The serum or plasma was separated and frozen.

Statistics: In each experiment, 4-5 animals were used per dose, with 6 to 8 animals as the control group that received the vehicle. The GH serum/plasma levels were averaged, the mean of the treated groups expressed in percent of the control group and the ID₅₀-value for the inhibition of the secretion was determined graphically (log-probit) for each experiment. In most experiments with enteral (intraduodenal) application only 1 dose, 10 mg/kg, was given. In order to be able to define at least an approximate ID₅₀, a scale was designed with a dose response curve expanding over 2 decades for inhibitions from 0 to 100%. Only inhibitions between 20 and 80% were considered reliable.

Hormone determination: GH was measured by radioimmunoassay (RIA)

| | | 2^{ND} ANTIBODY/ | | |
|---|---|---|---|---|
| HORMONE | ANTISERUM | SEPARATION | LABEL | STANDARD |
| GH | Monkey anti- | goat anti- | [¹²⁵I]rGH | NIAMD-rGH- |
| | rGH | monkey IgG | | RP1 |
| | | (Calbiochem, | | |
| | | 539873) | | |

Two to four experiments were performed. In the case of proper dose response curves, allowing the determination of an ID₅₀ values were averaged logarithmically. In the cases of single dose-experiments, the GH value in percent of control were averaged logarithmically and the ID₅₀ for this value was taken from the table. ID₅₀ values determined at the different time points during the infusion were averaged and this value was included in the tables.

Results

Examples of compounds with good in vivo results are summarized in Table 3. For reference, the respective results obtained with the natural SRIF 14 and Octreotride (Sandostatin®) are listed as well. Some showed comparable high binding affinities for the sst₂ subtype as measured for SRIF 14 or Octreotride (Sandostatin®), e.g. compound (+/−)-43, 46 or 47. In some cases, compounds were quite active when given by infusion but rather disappointing when given by the subcutaneous route. An obvious explanation for these discrepancies might be the lipophilicity of these compounds which results in a very slow absorption from the subcutaneous injection site.

TABLE 3
Effects of selected compounds in vitro (binding and GH inhibition) and in vivo in
the rat given by subcutaneous or intraduodenal routes and by infusion.

| | | | | GH vivo | | | |
|---|---|---|---|---|---|---|---|
| | Binding | | | | | Inf+ | Effect of |
| | | sst₂ | GH vitro | s.c. 1 h | i.d. 1 h | GHRH | 10 mg/kg |
| | Cortex | IC₅₀ | +GHRH | ID₅₀ | ID₅₀ | ID₅₀ | i.d. |
| Compound | IC₅₀ nM | nM | IC₅₀ nM | μg/kg | μg/kg | μg/kg/h | % of control |
| SRIF14 | 0.31 | 0.15 | 1.40 | 950.00 | | 3.90 | |
| Octreotride | 0.46 | 0.30 | 1.30 | 0.13 | 125 | 0.12 | |
| ac | | | | | | | |
| 45 dch | 0.75 | 3.00 | 5.10 | 48.00 | 11400 | 31.05 | 53 |
| 46 b | 0.66 | 0.43 | | ˜7.40 | Ø | 7.89 | 73 |
| | | | | | 19000 | | |
| 44 ch | 2.90 | 2.70 | 1.70 | 258.00 | 9721 | 43.00 | 48 |
| 48 tch | 0.99 | 2.50 | | | >> | 32.00 | 66 |
| | | | | | 15000 | | |
| (+/−)−43 ch | 0.37 | 0.28 | | 3.79 | 3417 | 3.72 | 32 |
| 47 ch | | 0.38 | | 10.00 | ˜454 | 8.10 | 45 |
| (+)−43 ch | | 0.18 | | 3.00 | 4800 | 1.68 | 36 |

The most active hydantoin by the parenteral route of administration was (+)-43 with ID₅₀ values of 3.0 and 1.68 μg/kg, if given subcutaneous or by infusion, respectively. (+/−)-43, which is also listed in Table 3, exhibited consistent inhibition of the GH secretion by 68% (ID₅₀=3.4 mg/kg) via enteral administration of 10 mg/kg. It was also very active in the in vitro binding assays (sst₂ affinity 3.0 nM) and in vivo when given by the parenteral route of administration. In the binding (sst₂) assay and in vivo during intravenous infusion, (+)-43 is clearly more active than the (+/−)-43, whereas after subcutaneous and after intraduodenal application similar activities were measured.

Compound 44 reached an enteral ID₅₀ below 10 mg/kg (ID50=9.7 mg/kg).

Compound 45 showed a reproducible inhibition of GH secretion in vivo (rat) after enteral application. The resulting ID₅₀ was 11.4 mg/kg.

Compound 46 exhibited parenteral activities at doses below 10 μg/kg (ID₅₀=7.4 μg/kg).

Compound 48 exhibited inhibition after 10 mg/kg given by the enteral route.

The most potent compound by the enteral route was compound 47, with a calculated ID₅₀ of 0.5 mg/kg. This value has been determined in a total of four independent experiments. This result indicated an improved enteral bioavailability of 1.8% (Octreotride:0.1%, intraduodenal 1 hour/infusion). However, in further studies compound 47 has also been tested by the enteral route of administration after repeated stimulation of the GH secretion by GHRH. In this model higher ID₅₀ values, in the range of 2.6 mg/kg (1 hour) were measured and the bioavailability was calculated to be 0.3%.

With most of these selected compounds additional in vivo experiments in other models (intraduodenal application followed by repeated stimulation of the GH secretion by GHRH, oral application by gavage of unanesthetized, estradiol primed rats) were performed. This data is tabulated in Tables 4 and 5. Table 4 summarizes data obtained for the inhibition of the stimulated GH secretion after subcutaneous, intraduodenal and intravenous application and the inhibitory effects obtained for different time points during intravenous infusion of selected hydantoins. The corresponding results for Octreotride (Sandostatin®) and for SRIF 14 are given as well.

For both, Octreotride (Sandostatin®) and (+/−)-43, a shorter duration of action has been found compared to subcutaneous application (Tables 4). With all compounds given intraduodenally (Table 4), a fairly regular decrease of activity from 15 minutes to 2 hours was found. The ID₅₀ values obtained on stimulated GH secretion are in most cases in the same range compared to results obtained after the same route of application on basal GH secretion. After intravenous infusion (Table 4), in most cases the apparent activity increases within time (decrease of ID₅₀ values). This corresponds to the assumption that the steady state plasma levels are only reached at the end of the infusion period.

TABLE 4
Effects of compounds on GH secretion in the rat in urethan anesthesia and with
repeated stimulation by intravenous application of GHRH (D-Ala₂GHRH₁₋₂₉NH₂, 1 μg/kg, 5
minutes, analogous to the infusion method). Comparison with respective inhibition of basal
secretion in ID₅₀ (wherein the ID₅₀ value is measured in μg/kg).

| | | | | | GH | | | | | |
|---|---|---|---|---|---|---|---|---|---|---|
| | | | | | BASAL | | | | | GH VIVO |
| | SUBCUTANEOUS + GHRH | | | | VIVO | INTRADUODENAL + GHRH | | | | BASAL |
| COMPOUND | 15′ | 30′ | 60′ | 120′ | S.C. 1 H | 15′ | 30′ | 60′ | 120′ | I.D. 1 H |
| Octreotide | 0.15 | 0.15 | 0.26 | | 0.13 | 24.2 | 16.6 | 26.8 | 82.0 | 125 |
| 45 | | | | | 48 | 4663 | 6520 | 14047 | ˜22000 | 11400 |
| 46 | | | | | 7 | | | | | 19000 |
| 44 | | | | | 258 | 13000 | 16000 | 19000 | | 9721 |
| 232-629 | | | | | | ø10000 | 17000 | 17000 | ø10000 | >>15000 |
| (+/−)−43 | 7.05 | 7.95 | 11.35 | 15.69 | 3.79 | 1165 | 1646 | 1419 | 2872 | 3417 |
| 235-575 | | | | | 10.30 | 1249 | ˜2420 | ˜2603 | 4500 | ˜454 |
| (+)−43 | | | | | 3.00 | | | | | 4800 |

| | | | | | I.V. SINGLE INJECTION + | | | |
|---|---|---|---|---|---|---|---|---|
| | I.V. SINGLE INJECTION + GHRH | | | | GHRH | | | INFUS. + GHRH |
| Compound | 15′ | 30′ | 60′ | 120′ | 15′ | 30′ | 60′ | MEAN |
| SRIF14 | | | | | 6.00 | 3.35 | 2.90 | 3.90 |
| Octreotide | | | | | 0.16 | 0.11 | 0.10 | 0.12 |
| 45 | | | | | 34.00 | 30.00 | 31.00 | 31.05 |
| 46 | | | | | 9.38 | 8.14 | 6.43 | 7.89 |
| 44 | | | | | 72.40 | 41.00 | 27.10 | 43.20 |
| 232-629 | | | | | 29.70 | 27.90 | 38.20 | 31.60 |
| (+/−)−43 | 12.50 | 31.20 | 55.00 | 124.10 | 6.44 | 3.58 | 2.24 | 3.72 |
| 235-575 | | | | | 10.30 | 6.90 | 7.30 | 8.06 |
| (+)−43 | | | | | 2.28 | 1.54 | 1.35 | 1.68 |

Table 5 summarizes the results obtained in the estradiol primed male rats that received the compounds orally by gavage. These rats have stabilized elevated GH plasma levels as well as increased prolactin levels. In contrast to normal untreated rats, who's prolactin secretion is not sensitive to the inhibitory effect of somatostain, in these rats the prolactin secretion is inhibited to a similar degree as the GH secretion. Therefore, the inhibitory effect can be measured on 2 different parameters, GH and prolactin. The mean enteral absorption and activity of the respective compounds are shown.

The results obtained (Table 5) confirm the results obtained in the other models, compound (+/−)-43 was the most active compound tested in this model with ID₅₀ values of 3 mg/kg (GH) and 1.8 mg/kg (prolactin).

TABLE 5
Effect of compounds in unanesthetized male rates bearing an
estradiol containing silastic implant. Application oral by gavage,
collection of blood sample from the retro-orbital plexus, 1 hour
after application. These effects were measured in ID₅₀ (wherein the
ID₅₀ value is measured in μg/kg)

| | GH | PROLA | |
|---|---|---|---|
| COMPOUND | 1 H | CT 1 H | MEAN |
| OCTREOTIDE | 440 | 998 | 663 |
| 45 | 8000 | 16000 | 11313 |
| 44 | 16000 | ø10000 | 16000 |
| 48 | 11000 | 9000 | 9950 |
| (+/−)−43 | 3000 | 1800 | 2324 |

The in vitro/in vivo studies on a large series of hydantoins have shown that non-peptidic SRIF agonist were identified with high affinity and potent activity on hormone release in vitro and in vivo. In vitro binding affinities and GH inhibiting effects in the range of the natural ligand SRIF 14 and of Octreotride (Sandostatin®) were achieved with quite a number of compounds

### EXAMPLE 5

Effects of compounds 44, 45 and 46 on Different Endocrine Parameters in the Rhesus Monkey.

Compounds 44, 45 and 46 are three selected hydantoins that showed SRIF-like agonistic activities (inhibition of the growth hormone secretion) in the rat after subcutaneous application. Therefore, they were further investigated in the Rhesus monkey and their activity profile was determined. Using subcutaneous administration, they were characterized in this species, by measuring their effects on the basal plasma levels of growth hormone, glucagon, insulin as well changes in glucose levels.

Compound 45 and 46 gave similar inhibitory profiles on hormone release as measured by Octreotride (Sandostatin®). The dose levels however necessary to achieve this effect were about 100 times higher than those determined for Octreotride (Sandostatin®). Compound 44 was weaker resulting in no consistent inhibitory effects up to 1100 μg/kg.

These non-peptidic agonists have different selectivity profiles in the Rhesus monkey from that found in the rat and/or a different inhibitory profile compared with peptides.

Methods

Animals and blood sampling: Rhesus monkeys, fed on the previous day in the morning with fruits only, were placed, slightly anesthetized with ketamin (Ketalar©, Parke-Davis), in primate chairs and brought to the experimental room. A catheter, consisting of an in-dwelling cannula (Vasocan Braunüle, 20G, B. Braun Melsungen AG, D-Melsungen), an extension line made from PP 800/110/260/100 Portex© tubing and a special adapter between cannula and tubing with a minimal void volume, was placed in a saphenic vein. The total volume of this catheter was approximately 0.7 mL. The distal end was placed through a hole in the wall to the adjacent room in order to allow infusion and blood sampling without being noticed by the animal. The animals were monitored with a video system.

During the entire duration of the experiment an infusion of 5 to 10 mL/h of isotonic saline (NaCl 0.9% Braun, B. Braun Medical AG, CH-Emmenbrücke) containing heparin sodium 80 mg/L (Biochemie GMBH, A-Kundl) was maintained, using a roller pump (Vario-Perpex, Guldener, CH-Zürich). At each sampling time 1.5 mL (twice the void volume of the catheter) was taken in one syringe before collecting the actual blood sample of 2 mL in another syringe. The first 1.5 mL were reinfused immediately after the actual sample had been taken. The sample was mixed with 0.1 mL of a mixture containing EDTA (ethlenediaminetetraacetic acid tetrasodium Fluka, CH-Buchs) and aprotinin (Trasylol©, Bayer) resulting in final concentrations of 1.8 mg/mL and 1000 KIE/mL, respectively. The blood samples were kept on ice and spun in a cooled centrifuge. The plasma was collected, divided in two aliquots and frozen: One aliquot was used for the determination of hGH, insulin and glucose, the other aliquot for glucagon. The remaining red blood cells were re-suspended in saline and kept cool until the termination of the experiment at which time they were reinfused.

Determinations: The blood levels of the hormones were determined by radioimmunoassay (RIA) using the appropriate antisera, antibodies, labels and standards, glucose by an enzymatic assay:

| | | 2^{ND} ANTIBODY/ | | |
|---|---|---|---|---|
| PARAMETER | ANTISERUM | SEPARATION | LABEL | STANDARD |
| hGH | rabbit anti-hGH | goat anti-rabbit | [¹²⁵I]hGH | Crescormon © |
| | (own) | (Calbiochem) | | Kabi AB, |
| | | | | Stockholm |
| Glucagon | rabbit anti- | goat anti- | [¹²⁵I]glucagon | included in kit |
| | glucagon | rabbit/PEG | | |
| | Kit: Glucagon | | | |
| | double antibody, | | | |
| | Diagnostic | | | |
| | Products | | | |
| | Corporation, | | | |
| | Los Angeles, | | | |
| | CA, USA | | | |
| Insulin | guinea pig anti- | goat anti-guinea pig | [¹²⁵I]insulin | Human, |
| | insulin | (Calbiochem) | (porcine) NEN | monocomp, |
| | | | | NOVO Biolabs |

Glucose: Hexokinase based method using the Aba-100 © bichromatic analyzer (Abbott)
Kit: a-gent ©, Glucose-UV, Abbott, CH-Zug Standard: Decision ©, Chemistry Control
Serum. Beckman Instr. Inc. Brea, CA, USA

Compounds: Compounds 44, 45 and 46 were dissolved in sterile water and dilutions were made using sterile isotonic glucose (5%) in order to administer doses of 1 to 100 μg/kg subcutaneous in a volume of 0.1 mL/kg. Four animals were treated with a given scheme or dose. The control group consisted of a total of four animals treated with the vehicle.

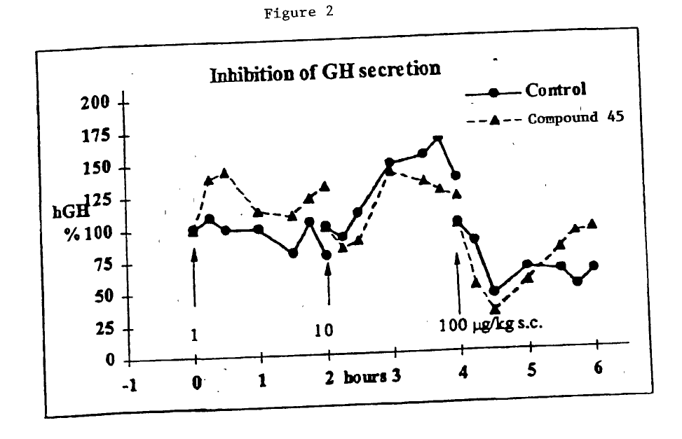

The study with compound 45 was done with consecutive applications of 1, 10 and 100 μg/kg subcutaneous at intervals of 2 hours, and the results are shown in FIG. 2.

Experimental scheme: Sixty minutes after arrival of the animals in the experiment room, and 30 minutes after insertion of the venous catheter, blood sampling was started. After 3 basal samples collected at 15 minutes intervals, the compounds were administered subcutaneously in the thigh. During the following 2 hours, the blood sampling interval of 15 minutes was maintained. Thereafter blood samples were taken every 30 minutes up to 6 hours. After the last samples had been taken, the erythrocytes collected duing the day were reinfused. The cannula was removed and the monkeys brought back to their quarters. In the pilot experiment with compound 45 blood was collected after 15, 30, 60, 90, 105 and 120 minutes. Immediately after the 2 hours sampling the next applications was given.

Statistics: The hormone and glucose levels of the first 3 samples, taken before each administration of the compound, were averaged logarithmically and this mean was taken as basal value. A log transformation was applied to individual values of plasma levels of the different parameters. It was found that this transformation renders the variances of the groups more homogenous and also leads to a more normal shape of the distributions. The standard error (SEM) of the logarithmic mean is a factor that gives the lower and upper 68% confidence limits, if the mean is divided or multiplied by this factor, respectively (H. P. Gubler).

In order to correct for the different basal levels, and to standardize the values, the means of the 3 basal values were set to 100% and the values at the different time points are expressed in percent of the basal values. This set of data was used for the graphical presentation. In order to calculate an ID₅₀, the percent values calculated for the time points 30 minutes to 2 hours were averaged logarithmically and the ID₅₀ determined graphically on log/probit paper.

### EXAMPLE 6

Brain Penetration in Rat after IV Infusion

The in vivo brain penetration of compound 42 after a 48-hour intravenous infusion was assessed. [¹⁴C]-42-ch was intravenously administered to 5 male rats at a loading dose of 0.3 mg(-b)/rat and then infused at a constant rate of 100 μg(-b)/h for 48 hours using a syringe pump connected to the femoral vein. Immediately after stopping the infusion, the rats were sacrificed. Blood and brain were collected and analyzed for total radioactivity and unchanged compound 42.

After a 48-hours intravenous infusion of 100 μg/h, the blood concentration of compound 42 was 66±14 ng/mL; the concentration, determined in brain amounted to 18±7 ng/g. The observed brain/blood ratio of 0.27±0.07 indicates a low but significant brain penetration of this compound after intravenous infusion.

Concentrations (ng/g) of 42 in blood and brain of rats at 48
hours after intravenous infusion

| | ANIMAL NO. | | | | | |
|---|---|---|---|---|---|---|
| TISSUE | 1 | 2 | 3 | 4 | 5 | MEAN ± SD |
| Blood | 59 | 76 | 76 | 73 | 45 | 66 ± 14 |
| Brain | 16 | 21 | 29 | 16 | 10 | 18 ± 7 |
| Brain/Blood Ratio | 0.27 | 0.27 | 0.38 | 0.21 | 0.22 | 0.27 ± 0.07 |

### EXAMPLE 7

Effect of compound 14 on Rat Brain SRIF Receptors

Ex-vivo binding was used to determine to whether compound 14 (a potent sst₂ receptor selective hydantoin) when applied peripherally to rates can cross the blood brain barrier, as robust behavioral models are not fully characterized and blood-brain penetration models are not routinely available. These results were compared to the [¹²⁵I]-labeled Tyr³ analogue of Octreotride™, which labels predominantly SS-1 sites (sst₂ receptors) and [¹²⁵]Tyr²⁶-SRIF-28 which in principle recognizes all SRIF receptors.

Compound 14 or a saline control (5 mL/kg) was applied sub-continuously to Wistar rats (200 g, 3 per group) at 0.3, 1, 3 and 10 mg/kg and the effects on cerebral cortex and hippocampus binding measured 60 minutes after application. The rats were given saline or drug sub-cutaneously at the indicated dose (in saline) and killed 60 minutes after application with CO₂. The brain was removed and placed on ice; the cerebral cortex and hippocampus were dissected out and weighted. The tissue was homogenized for 15 seconds in ice cold buffer (Hepes 10 mM, PH 7.5., BSA 0.5%) at Ig/40 mL (cortex) or 1 g/60 mL (hippocampus). The homogenate was stored on ice and used immediately or deep frozen (−70° C.) until used (1-3 days).

SS-1/sst2 binding studies: 150 μL of rat brain membranes were incubated in 96 well plates for 60 minutes at 22° C. in 10 mmol/L HEPES (pH 7.6) containing 5 mmol/L MgCl₂, 10 mg/mL bacitracin and 0.5% (W/V) bovine serum albumin, 50 μL [¹²⁵I] labeled Tyr³ analogue of Octreotride™ (2175 Ci/mmol, 25-50 pmol/L final concentration and 50 μL of buffer without (total binding) or with 1 μM SRIF-14 (non specific binding). The binding reaction was started by the addition of membranes and stopped after 60 minutes by rapid washing with 5 mL of ice cold Tris HCl 10 mM, NaCl 154 mM, pH 7.5 buffer (two times) and rapid filtration over glass fiber filters (preincubated with 0.3% polyethyleneimine to reduce non specific binding). The filters were dried and counted in a Wallac Beta plate counter.

[¹²⁵I]Tyr²⁶-SRIF-28 binding: was performed as described above for SS-1/sst2 binding with 25-50 pM of either ligand.

Dose-Dependency:

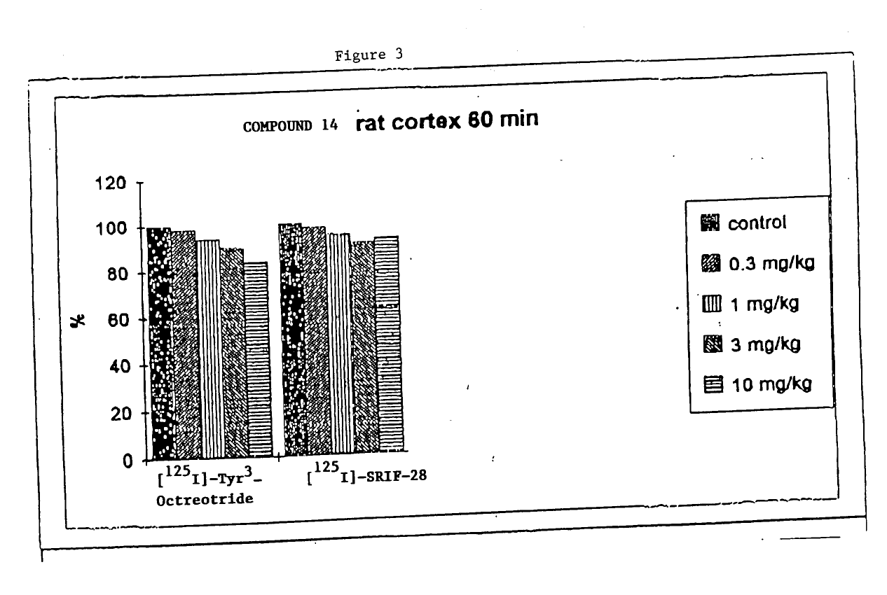

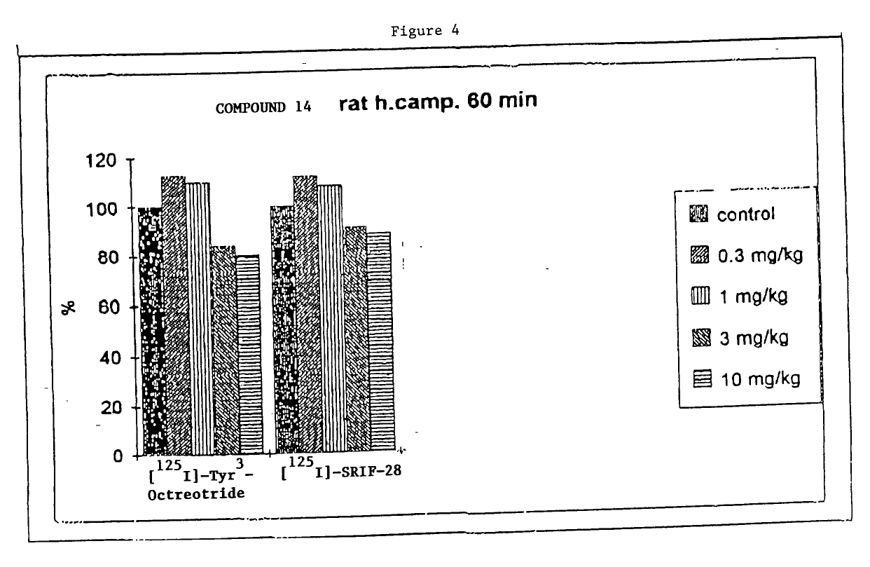

A summary of the data obtained is tabulated in Tables 6 through 9 and FIGS. 3 and 4. The [¹²⁵I]-labeled Tyr³ analog of Octreotride binding in cortex and hippocampus was slightly but dose-dependently affected. At 10 mg/kg there was a maximal decrease in the 20% range. [¹²⁵I]SRIF-28 binding (in the presence of 5 mM MgCl₂) revealed also a limited decrease in binding. Therefore, SRIF binding was dose-dependently reduced in both cortex and hippocampus 60 minutes following application. However, the decrease in binding was very limited and maximal effect of about 20% was obtained for sst₂ (SS-1) binding at 10 mg/kg subcutaneous The effects on other binding sites appear to be negligible.

In conclusion, compound 14 when applied subcutaneously for 60 minutes does slightly affect cortical and hippocampal binding of [¹²⁵I]-labeled Tyr³ analogue of Octreotride™. SRIF-28 binding appears to be affected at the highest doses as well. This data suggests that compound 14 crosses the blood brain barrier and reaches both the cortex and hippocampus, particularly at high doses, and is a selective sst₂ inhibitor. This is compatible with the sst₂ selectively of this ligand. The degree of receptor occupancy is limited, although in well coupled systems, 20% receptor occupancy can be sufficient to lead to significant receptor activation. In an effectively coupled receptor effect system, such a level of occupancy may be sufficient to significantly activate receptors (e.g. SS-1 binding: pKd=8.56, sst2 cyclase: pEC₅₀=9.32). In addition, when applied subcutaneous 14 inhibited GH release with an EC₅₀ in the 1 mg/kg range.

The tables list the effects of compound 14 on cortex (Table 6) and hippocampus (Table 7) binding at the doses and times indicated (see also FIGS. 3 and 4). TB=total binding, NS=non specific binding, SB=specific binding. The values are indicated in cpm for individual animals, mean, n=number of animals, sem=standard error of the mean; percent=% specific binding remaining following treatment compared to controls.

TABLE 6

| | | | | 14 | | | 14 | | | 14 | | | 14 | | |
|---|---|---|---|---|---|---|---|---|---|---|---|---|---|---|---|
| cortex | CONTROL | | | 0.3 mg/kg | | | 1 mg/kg | | | 3 mg/kg | | | 10 mg/kg | | |
| 60 min | TB | NS | SB | TB | NS | SB | TB | NS | SB | TB | NS | SB | TB | NS | SB |
| Tyr³-Oct | | | | | | | | | | | | | | | |
| 26122 | 25222 | 2953 | 22269 | 25180 | 2753 | 22427 | 22345 | 2623 | 19722 | 22963 | 2519 | 20444 | 22052 | 2527 | 19525 |
| | 26165 | 3024 | 23141 | 23891 | 2634 | 21257 | 26063 | 2705 | 23378 | 22335 | 2511 | 19624 | 20915 | 2575 | 18340 |
| | 26122 | 2776 | 23346 | 26474 | 2685 | 23789 | 24466 | 2642 | 21824 | 24259 | 2559 | 21700 | 22380 | 2740 | 19640 |
| mean | 25836 | 2918 | 22919 | 25182 | 2691 | 22491 | 24298 | 2657 | 21641 | 23186 | 2530 | 20658 | 21782 | 2614 | 19168 |
| n | 3 | 3 | 3 | 3 | 3 | 3 | 3 | 3 | 3 | 3 | 3 | 3 | 3 | 3 | 3 |
| sem | 307 | 74 | 330 | 746 | 34 | 732 | 1082 | 25 | 1059 | 566 | 15 | 552 | 444 | 65 | 415 |
| percent | | | 100 | | | 98 | | | 94 | | | 90 | | | 84 |
| SRIF-28 | | | | | | | | | | | | | | | |
| +MgCl-2 | 27439 | 3290 | 24149 | 27376 | 3058 | 24318 | 24682 | 2915 | 21767 | 25171 | 2980 | 22191 | 25800 | 3070 | 22730 |
| | 28386 | 3591 | 24795 | 26208 | 2970 | 23238 | 26966 | 3036 | 23930 | 25008 | 3016 | 21992 | 24410 | 3015 | 21395 |
| | 27227 | 3176 | 24051 | 26696 | 3027 | 23669 | 27096 | 3191 | 23905 | 25694 | 3116 | 22578 | 26910 | 3305 | 23605 |
| mean | 27664 | 3352 | 24332 | 26760 | 3018 | 23742 | 26248 | 3047 | 23201 | 25291 | 3037 | 22254 | 25707 | 3130 | 22577 |
| n | 3 | 3 | 3 | 3 | 3 | 3 | 3 | 3 | 3 | 3 | 3 | 3 | 3 | 3 | 3 |
| sem | 356 | 124 | 233 | 339 | 26 | 314 | 784 | 80 | 717 | 207 | 41 | 172 | 723 | 89 | 643 |
| percent | | | 100 | | | 89 | | | 95 | | | 91 | | | 93 |

| | | | | | |
|---|---|---|---|---|---|
| cortex | CONTROL | 14 | 14 | 14 | 14 |
| 60 min | | 0.3 mg/kg | 1 mg/kg | 3 mg/kg | 10 mg/kg |

TABLE 7

| | | | | 14 | | | 14 | | | 14 | | | 14 | | |
|---|---|---|---|---|---|---|---|---|---|---|---|---|---|---|---|
| h. camp. | CONTROL | | | 0.3 mg/kg | | | 1 mg/kg | | | 3 mg/kg | | | 10 mg/kg | | |
| 60 min | TB | NS | SB | TB | NS | SB | TB | NS | SB | TB | NS | SB | TB | NS | SB |
| Tyr³-Oct | | | | | | | | | | | | | | | |
| +MgCl-1 | 14140 | 2460 | 11680 | 14682 | 2116 | 12566 | 14192 | 2144 | 12048 | 13031 | 1859 | 11172 | 12945 | 1886 | 11059 |
| | 14208 | 2454 | 11754 | 15597 | 2294 | 13303 | 16963 | 1953 | 15010 | 11216 | 1984 | 9232 | 12119 | 2078 | 10041 |
| | 16285 | 2292 | 13993 | 18430 | 2092 | 16338 | 16013 | 1913 | 14100 | 12939 | 1959 | 10980 | 10974 | 2037 | 8937 |
| mean | 14878 | 2402 | 12476 | 16238 | 2167 | 14069 | 15723 | 2003 | 13719 | 12395 | 1934 | 10461 | 12013 | 2000 | 10012 |
| n | 3 | 3 | 3 | 3 | 3 | 3 | 3 | 3 | 3 | 3 | 3 | 3 | 3 | 3 | 3 |
| sem | 704 | 55 | 759 | 1128 | 64 | 1154 | 813 | 71 | 876 | 590 | 38 | 617 | 571 | 58 | 613 |
| percent | | | 100 | | | 113 | | | 110 | | | 84 | | | 80 |
| SRIF-28 | | | | | | | | | | | | | | | |
| +MgCl-2 | 19027 | 2978 | 16049 | 21155 | 2936 | 18219 | 19809 | 2754 | 17055 | 18290 | 2532 | 15758 | 18398 | 2587 | 15809 |
| | 18776 | 3034 | 15742 | 20998 | 2874 | 18124 | 21044 | 2528 | 18516 | 17117 | 2841 | 14336 | 17237 | 2850 | 14387 |
| | 20693 | 2832 | 17861 | 22072 | 2753 | 19319 | 20766 | 2621 | 18145 | 17948 | 2786 | 15162 | 18426 | 2711 | 13715 |
| mean | 19499 | 2948 | 16551 | 21408 | 2854 | 18554 | 20540 | 2634 | 17905 | 17805 | 2720 | 15085 | 17353 | 2716 | 14637 |
| n | 3 | 3 | 3 | 3 | 3 | 3 | 3 | 3 | 3 | 3 | 3 | 3 | 3 | 3 | 3 |
| sem | 602 | 60 | 661 | 335 | 54 | 383 | 374 | 66 | 438 | 329 | 95 | 412 | 572 | 76 | 617 |
| percent | | | 100 | | | 112 | | | 108 | | | 91 | | | 88 |

| | | | | | |
|---|---|---|---|---|---|
| h. camp. | CONTROL | 14 | 14 | 14 | 14 |
| 60 min | | 0.3 mg/kg | 1 mg/kg | 3 mg/kg | 10 mg/kg |

TABLE 8

| | | | | | | | | | | | | | | GH, UR |
|---|---|---|---|---|---|---|---|---|---|---|---|---|---|---|
| | Bind | Bind | | | | | | | | | GH, Ur | GH, UR | GH, UR | Inf |
| | SS1 | −090 | Bind | Bind | Bind | Bind | Bind | GH | GH sc | GH id | Inf15′ | Inf30′ | Inf60′ | Mean |
| | D.H. | Cortex | SSTR1 | SSTR2 | SSTR3 | SSTR4 | SSTR5 | vitro | 1 h | 60′ | +GHRH | +GHRH | +GHRH | +GHRH |
| Cmpd | nM | nM | nM | nM | nM | nM | nM | nM | μg/kg | μg/kg | μg/kg/h | μg/kg/h | μg/kg/h | μg/kg/h |
| SRIF 14 | 0.07 | 0.31 | 0.5 | 0.15 | 0.3 | 1.2 | 0.59 | 1.4 | 950.00 | | 6.000 | 3.300 | 2.900 | 3.900 |
| Octreotride | 0.30 | 0.46 | >1000 | 0.30 | 30.0 | >1000 | 7.20 | 1.3 | 0.13 | 125 | 0.124 | 0.082 | 0.085 | 0.096 |
| ac | | | | | | | | | | | | | | |
| 42 ch | | 11.00 | 50000.0 | 44.00 | 2800.0 | 5300.0 | 1400.00 | 255.0 | | Ø10000 | 856.000 | 434.000 | 425.000 | 540.000 |
| 14 ch | 2.50 | 2.70 | >1000 | 5.70 | 300.0 | >1000 | 450.00 | 11.0 | 1160.00 | >30000 | ˜44 | 11.000 | 14.000 | 19.000 |
| 45 ch | 1.00 | 0.75 | 1400.0 | 3.00 | 220.0 | >1000 | 97.00 | 5.1 | 48.00 | 11400 | 34.000 | 30.000 | 30.000 | 30.000 |
| 46 b | 0.63 | 0.66 | >1000 | 0.43 | 450.0 | >1000 | 90.00 | | | Ø10000 | 9.400 | 8.100 | 6.400 | 7.900 |
| 44 ch | 1.00 | 2.90 | >1000 | 2.70 | >1000 | >1000 | 170.00 | *1,7 | | 9721 | | | | |

Ø = no effect at this dose > = extrapolated value

TABLE 9

| | | | | | | | | | | | | | | Mean |
|---|---|---|---|---|---|---|---|---|---|---|---|---|---|---|
| | Bind | Bind | | | | | | | | | GH, Ur | GH, UR | GH, UR | GH, UR |
| | SS1 | −090 | Bind | Bind | Bind | Bind | Bind | GH | GH sc | GH id | Inf15′ | Inf30′ | Inf60′ | Inf |
| | D.H. | Cortex | SSTR1 | SSTR2 | SSTR3 | SSTR4 | SSTR5 | vitro | 1 h | 60′ | +GHRH | +GHRH | +GHRH | +GHRH |
| Cmpd. | nM | nM | nM | nM | nM | nM | nM | nM | μg/kg | μg/kg | μg/kg/h | μg/kg/h | μg/kg/h | μg/kg/h |
| SRIF 14 | 0.07 | 0.31 | 0.5 | 0.15 | 0.3 | 1.2 | 0.59 | 1.4 | 950.00 | | 6.000 | 3.300 | 2.900 | 3.900 |
| octreotride | 0.30 | 0.46 | >1000 | 0.30 | 30.0 | >1000 | 7.20 | 1.3 | 0.13 | 125 | 0.124 | 0.082 | 0.085 | 0.096 |
| ac | | | | | | | | | | =0.10% | | | | |
| 42 ch | | 11.00 | 50000.0 | 44.00 | 2800.0 | 5300.0 | 1400.00 | 255.0 | | Ø10000 | 856.000 | 434.000 | 425.000 | 540.000 |
| 14 ch | 2.50 | 2.70 | >1000 | 5.70 | 300.0 | >1000 | 450.00 | 11.0 | 1160.00 | >30000 | ˜44 | 11.000 | 14.000 | 19.000 |
| 45 dch | 1.00 | 0.75 | 1400.0 | 3.00 | 220.0 | >1000 | 97.00 | 5.1 | 48.00 | 11400 | 34.000 | 30.000 | 30.000 | 30.000 |
| | | | | | | | | | | =0/42% | | | | |
| 46 b | 0.63 | 0.66 | >1000 | 0.43 | 450.0 | >1000 | 90.00 | | | Ø10000 | 9.400 | 8.100 | 6.400 | 7.900 |
| 44 ch | 1.00 | 2.90 | >1000 | 2.70 | >1000 | >1000 | 170.00 | *1,7 | | 9721 | 50 | 30 | 21 | 32 |

Ø = no effect at this dose > = extrapolated value

### EXAMPLE 8

Absorption and Disposition In Rat

The absorption and disposition characteristics of [¹⁴C]-42 in rats after oral (10 mg/kg) and intravenous (1 mg/kg) administration with a one week interval between doses was analyzed. Moreover, the brain penetration and the in vitro blood distribution and plasma binding was investigated.

The labeling of 42 with carbon-14 was carried out by the Isotope Laboratories of Sandoz Pharma, Basel, and its purity was checked by HPLC. UV- and radioactivity evaluation of the chromatograms showed similar chemical purity as that of the reference standard and a radiochemical purity of >98%. The labeled compound had a specific radioactivity of 72 μCi/mg (-ch). All dose levels and concentrations given from hereon refer to the free base form of the compound.

The pharmacokinetic study was performed with 3 male Wistar rats (BRL) weighing 300-330 g with a one week interval between administration phases. The day before the first dosing, the rats underwent surgical implantation of an in-dwelling cannula; the right femoral artery was cannulated and the tube was passed subcutaneously to emerge at the back of the neck. The animals were individually housed in metabolism cages and fasted overnight before administrations.

For the oral dose (10 mg(-b)/kg or 720 μCi/kg), [¹⁴C]-42-ch was dissolved in ethanol-water (1:9 v/v) at the concentration of 2 mg(-b)/mL. The dose solution was administered (5 mL/kg) by gastric incubation.

For the intravenous dose (1 mg(-b)/kg or 72 μCi/kg), [¹⁴C]-42-ch was dissolved in ethanol-saline (1:16 v/v) at a concentration of 0.5 mg(-b)/mL. The dose solution was administered (2 mL/kg) into the surgically exposed femoral vein.

During the study, 18 blood samples (100-500 μL) were taken, up to 48 hours post-dose from the cannulated femoral artery representing a total volume of 5.7 mL blood. The loss of blood was compensated by infusion of 6 times 1 mL of blood from donor rats. Urine samples were collected up to 48 hours post-dose. For the analysis of the in vivo brain distribution, 3 male rats were intravenously dosed (1 mg/kg or 72 μCi/kg) as described above. At 0.5 hours after administration, the rats were sacrificed; blood and brain were collected.

The unidirectional influx for [¹⁴C]-42 was measured by the brain sampling single injection technique in adult male Wistar rats (˜220 g) under anesthesia (ketamine 130 mg/kg i.m., xylazine 1.3 mg/kg i.m.) (Oldendorf, W. H. “Measurement of brain uptake of radiolabeled substances using tritiated water internal standard” Brain Res., 1970, 24, 372-376). A bolus of ˜220 μL 0.001 M HEPES-buffered Ringer's solution pH 7.4, or rat plasma, was rapidly injected into the common carotid artery. The bolus contained [¹⁴C]-42 (68 μg/mL) together with tritiated water (25 μCi/mL); the amount of ethanol in the injectate was 1% (v/v). The animals were decapitated 5 seconds after the injection. Samples of the injection solution and the brain hemisphere ipsilateral to the injection side were solubilized in 2 mL soluene-350 (Packard) at room temperature for the night before double isotope liquid scintillation counting. The percentage BUI (Brain Uptake Index) was calculated as

100*(¹⁴C/³H dpm)_{brain}/(¹⁴C/³H dpm)_{injectate}

The brain extraction ratio E was calculated from E=BUI*0.62, where 0.62 represents the brain extraction ratio of tritiated water (Pardridge, W. M. et al. “Absence of albumin receptor on brain capillaries in vivo or in vitro” Am. J. Physiol, 1985, 249, E264-E267). In order to quantify a possible sequestration of [¹⁴C]-42 by the brain microvasculature, the brain hemispheres of 3 rats were submitted to capillary depletion (Triguero, D, et al. “Capillary depletion method for quantifying the blood-brain barrier transcytosis of circulating peptides and plasma proteins” J. Neurochem, 1990, 54, 1882-1888). The ¹⁴C-radioactivity observed in the pellet (capillary bed/endothelial cells and pericytes) was compared to the ¹⁴C-radioactivity observed in the supernatant (transcytosis space/interstitial fluid).

In vitro blood distribution and plasma protein binding. For blood distribution studies, fresh, heparinized rat blood (n=3) was spiked with [¹⁴C]-42 to achieve the final concentrations of 5, 50, 500 and 5000 ng/mL. After a 30-minutes incubation at 4° C., 22° C. and 37° C., the samples were centrifuged (1600×g, 10 minutes, at the incubation temperature) to get plasma. The fraction free of 42 in plasma of rat and human was determined by equilibrium dialysis. Phosphate buffer was spiked with [¹⁴C]-42 (50, 500, 5000 ng/mL) and dialyzed versus blank rat and human plasma at 37° C. for 2 hours. The radioactivity was determined in spiked blood samples, in plasma obtained after centrifugation and in both compartments after dialysis.

Determination of radioactivity in blood, plasma, brain homogenates and in urine, was carried out by direct liquid scintillation counting. Prior to radiometric determination, blood and tissue were solubilized in Solutron (Kontron Instruments, Zurich, Switzerland). After adding 10 mL of scintillation cocktail (Lumasafe, Lumac, Landgraaf, the Netherlands), all samples were counted in Tri-carb liquid scintillation analyzer (Canberra Packard, IL). Automatic external standard techniques (quench compensation) were employed to determine the efficiency of the respective radiometric analyses; observed data (counts per minute, cpm) were converted to disintegrations per minute (dpm).

The concentration of [¹⁴C]-42 in the whole blood samples was determined by LC-RID (liquid chromatography—reversed isotope dilution). The procedure involved the addition of 5 μg of non-radiolabeled 42 to each blood sample as an internal standard. After adding 1 mL of acetonitrile, the sample was mixed with a Polytron mixer and centrifuged (234000×g, 30 minutes) in a Beckman centrifuge (Model TL100). The supernatant was evaporated in a vacuum centrifuge (Univapo 150H, Zivy). The residue was reconstituted in 250 μL of mobile phase-water (3:1 v/v) and centrifuged (3000×g, 60 s). The supernatant (200 μL) was injected onto HPLC. (MT2, Kontron Instruments). Compound 42 was separated from potential metabolites and endogenous compounds on a RP18 endcapped Superspher column, 125 mm×4 mm (Merck) at 45° C. The mobile phase consisted of 0.1% tetramethylammonium hydroxide—acetonitrile (500:500 v/v). The flow rate was 1 mL/min; the effluent was monitored at 260 nm. The peak corresponding to the unchanged [¹⁴C]-42 was collected in a polyethylene vial by a fraction collector (SuperFrac, Pharmacia LKB) and subjected to radioactivity determination. The concentration of [¹⁴C]-42 in each sample was calculated from the ratio of the amount of radioactivity in the eluate fraction corresponding to 42 and the area of the ultraviolet absorbance of the non-radiolabeled 42 used as an internal standard. Recoveries averaged 87±15%. The limit of quantification was 0.3 ng/mL.

Metabolite patters were determined in blood extracts and in urine. Blood was pooled from the 3 intravenously dosed rats of the pharmacokinetic study and from 3 additional rats treated in the same way. Urine was obtained from the animals of the pharmacokinetic study.

Time-pools of blood were prepared from samples that had been diluted with a two-fold volume of water and hence at least partially hemolyzed before storage at −20° C. Between 0.3 and 0.6 mL of pooled diluted blood were spiked with 10-20 μg unlabeled compound 42-ch, extracted with 10 mL of methanol (HPLC grade, Rathburn) by sonication and centrifuged. The pellet was extracted once more in the same way. The two supernatants were combined and evaporated under reduced pressure at 35° C. on a rotary evaporator. The residue was transferred into a smaller vial by means of methanol and water, evaporated under a stream of nitrogen and taken up in 80 μl methanol and 320 μl water or in 120 μl methanol and 280 μl water by sonication. The suspension was centrifuged and a 300 μl aliquot of the supernatant was injected onto the HPLC column. The extraction yield of radioactivity was only 45±12% (mean ISD).

To urine samples, acetonitrile (˜5% v/v), TFA (to obtain a pH of ˜3) and unlabeled compound 42-ch (˜4 μg per volume analyzed) were added. The mixtures were centrifuged and 1-2 mL of supernatant were injected onto the HPLC column.

The chromatography was performed using HP 1090 liquid chromatograph (Hewlett-Packard). The radioactivity of the column eluate was measured by liquid scintillation counting either off-line or on-line using a Berthold LB 507A radioactivity monitor. The samples were chromatographed on a reversed-phase column (Nucleosil 100, C18AB, 250×4.6 mm, 5 μm particle size, Macherey-Nagel) protected by a corresponding 8×4 mm precolumn. The column temperature was 40° C. The components were eluted with a gradient of 0.02% v/v trifluoroacetic acid (TFA, Pierce) in water (mobile phase A, pH 2.8) versus acetonitrile (HPLC grade S, Rathburn; mobile phase B). The proportion of solvent B was kept at 5% up to 5 minutes after injection and was then increased in linear segments to 40% at 110 minutes and 100% at 120 minutes where it was kept for another 10 minutes. The total flow rate was 1 mL/min. The unlabeled parent drug, added as a retention time marker, was monitored by UV detection at 216 nm.

The peeling method was applied to describe the data by a compartment model approach, characterized by the following equation: C═C₁*e^{λ1-t}+C₂*e^{λ2-t}. The initial estimates of C₁, λ₁, C₂, λ₂ were taken to generate the best fit using the computer software ELSFIT. The estimates for half-lives were calculated as T_{1/2λi}=ln2/λᵢ. Areas under the curve (AUC) and areas under the first-moment curve (AUMC) were calculated by the trapezoidal rule and extrapolated to infinite time. The fraction of elimination associated with the final exponential term f₂ was calculated as (C₂,/λ₂)/Area. Total clearance (CL) was calculated as Dose/AUCᵢᵥ. The volume of distribution at steady state was calculated as Vₛₛ=MRT*CL, where MRT is the mean residence time, calculated at AUMC/AUC.

Results and Discussion

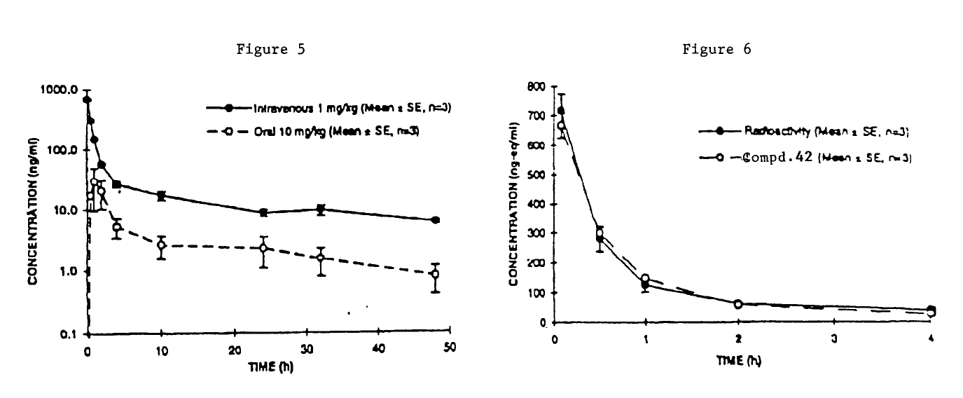

Absorption. Based on the AUC ratios for total radioactivity, the absorption of drug derived radioactivity was 2.2±1.8%. Considering AUC ratios p.o./intravenous for parent drug, an average bioavailability of 1.3±0.9% was estimated (Tables 10-12; FIGS. 5 and 6).

Disposition. Compound 42 is distributed to tissues as demonstrated by the large volume of distribution (Vₛₛ=20 l/kg). The elimination (CL=4.5 mL/min) occurred essentially by hepatic clearance (only 3% of dose was recovered in urine, although urine was only semiquantitatively collected). After intravenous bolus, the concentration of compound 42 in blood declined biphasically with a first half-life of 0.45 h (t_{1/2λ1}) and a terminal half-life of 24 h (t_{1/2}). The fact that f₂ (fraction of dose eliminated within λ₂) was 67% means that the majority of drug was eliminated within the terminal phase.

Brain Penetration. At 0.5 hours after an intravenous bolus, no significant brain penetration of [¹⁴C]-42 could be demonstrated (Table 13). The radioactivity concentration ratio brain/blood amounted to 0.03, which corresponds roughly to the brain contamination by vascular blood. However, the unidirectional brain extraction of [¹⁴C]-42, obtained by the BUI experiments (Table 14) was high; 41±0.9% when Ringer buffer was used as a vehicle solution and very low: 3±1% with addition of rat plasma. The capillary depletion experiments (n=3 rats) indicated that the fraction taken up by capillaries represented only 5% of the brain penetration, suggesting an insignificant uptake of compound 42 by brain capillary endothelium. Thus the BUI experiments indicated a fairly high passage of compound 42 in the absence of plasma protein binding. Nevertheless, because of its protein binding, the brain extraction of this compound is strongly reduced in the presence of plasma proteins to the insignificant value of 3%.

Blood Distribution and Plasma Protein Binding. The blood distribution of compound 42 is slightly concentration dependent within the range investigated (5-5000 ng/mL). In addition, temperature dependency was observed. The proportion of compound 42 present in plasma was 40-60% at 37° C. and 69-92% at 4° C. Within the investigated concentration range (50-5000 ng/mL), the fraction free in rat plasma was constant, ranging between 10-12%. The fraction free in human plasma was 15-23%, showing a concentration dependency between 50-500 ng/mL (Tables 15 and 16).

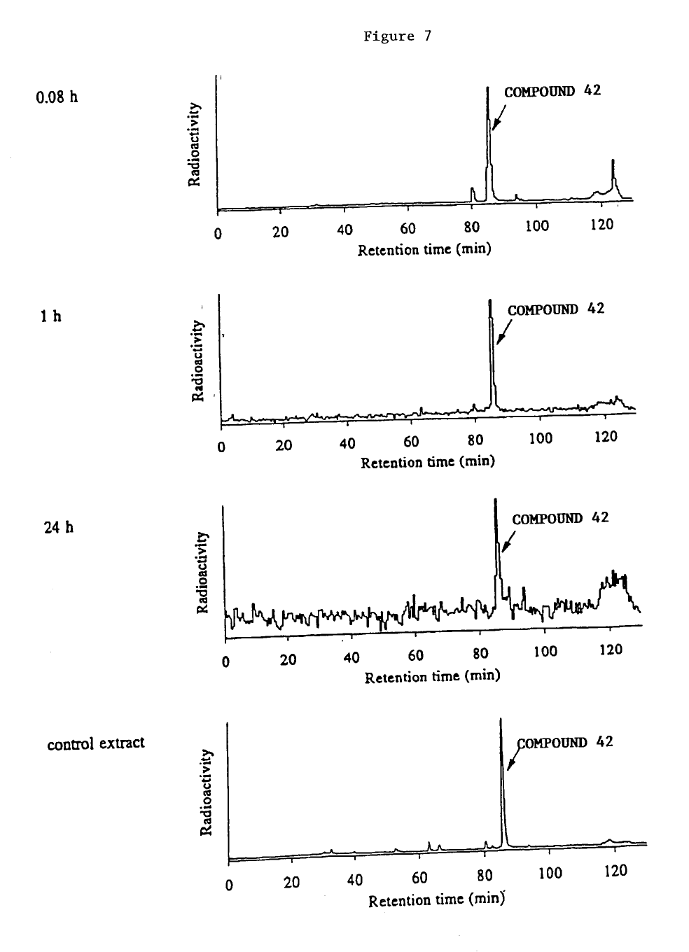

Metabolism. Radiochromatograms of blood extracts after intravenous dosing and of a control extract are shown in FIG. 7. After p.o. dosing, the blood did not contain enough radioactivity for obtaining metabolite patterns. Parent drug formed the highest peak in the chromatograms. Part of the minor peaks at 80 and 94 minutes retention time might represent metabolites of compound 42, as concluded from the patterns in urine (see below), but the two peaks appeared to some extent also in the chromatogram of the control extract. The broad hump around 120 minutes is probably an artifact. Different amounts of this nonpolar material were observed depending on the extraction procedure and the chromatographic conditions. Therefore, these components were formed both during the sample preparation and during chromatography. Especially large amounts were produced on acidifying for a short time blood extracts with TFA to pH ˜2 but not on acidifying in the same way a fresh solution of the compound in water/ethanol. During storage of a stock solution of compound 42 in water/ethanol at −20° C., nonpolar degradation products were formed also, but at a low rate. The nature of these components, and whether those formed in the presence and in the absence of biological material are identical, remains to be investigated. The late eluting material does not seem to represent unchanged drug (retained on the column), as demonstrated by isolation and re-analysis. In conclusion, the drug-related material in blood consisted mainly of unchanged drug.

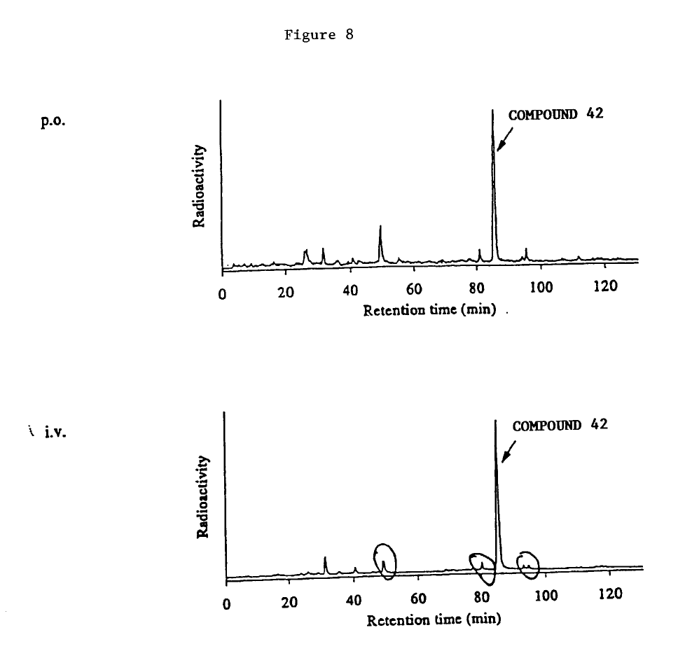

The amounts of total radioactivity and parent drug found in urine are given in Table 17. Even though these numbers represent underestimates because some urine was lost during blood sampling, it is clear that urinary excretion of drug-related material is very minor. Examples of radiochromatograms are given in FIG. 8. After intravenous dosing, unchanged compound 42 dominated the patterns in urine. After oral dosing, the patterns showed large individual differences. The peaks at 49, 80, 94 and 95 minutes retention time varied in parallel with the signal of the parent drug and with the extent of absorption of radioactivity Table 10A). These peaks, therefore, seem to represent true metabolites of compound 42, whereas the others rather represent impurities of the radiolabeled drug (with higher urinary excretion and/or higher absorption than the drug) or metabolites thereof.

Compound 42 is distributed to tissues (Vₛₛ=20 l/kg). The unidirectional brain extraction (Brain Uptake Index) of 42 is high (41%); however the binding of this compound to plasma proteins (−90%) reduces this brain penetration. The AUC ratio parent drug/radioactivity observed after intravenous administration amounts to 0.7. Metabolite patterns also confirm that the drug-related material in blood represents mainly unchanged drug. The elimination is relatively slow (t_{1/2}=24 h) and the systemic clearance (4.5 mL/min) consists essentially in hepatic clearance.

The low oral bioavailability of compound 42 (1.3±0.9%) may be attributed to a poor absorption and not to a presystemic first-pass effect. Additional information on the low absorption and brain penetration of compound 42 will be obtained by means of in vitro studies with Caco-2 cells and bovine brain capillary endothelial cells.

Compound 42 is distributed into tissues (Vₛₛ=20 l/kg). The unidirectional brain extraction (Brain Uptake Index) of compound 42 is significant (41%).

The AUC ratio parent drug/radioactivity of 0.7 and the metabolite patterns show that the drug-related material in blood represents mainly parent drug.

TABLE 10A
Radioactive concentration (ng-eq/mL) in blood after oral
administration of 10 mg/kg [¹⁴C]-42

| TIME | ANIMAL NO. | | | | |
|---|---|---|---|---|---|
| (h) | 01 | 02 | 03 | MEAN | SD |
| 0.5 | 1.1 | 19.2 | 26.9 | 15.7 | 13.3 |
| 1 | .08 | 12.8 | 19.3 | 10.9 | 9.4 |
| 2 | 1.1 | 13.3 | 16.8 | 10.4 | 8.2 |
| 4 | 0.8 | 10.7 | 12.2 | 7.9 | 6.2 |
| 8 | 0.4 | 6.4 | 8.6 | 5.1 | 4.2 |
| 24 | 0.7 | 6.3 | 7.7 | 4.9 | 3.7 |
| 32 | 0.2 | 4.9 | 7.3 | 4.1 | 3.6 |
| 48 | 0.1 | 4.3 | 3.2 | 2.5 | 2.2 |
| AUC (ng-eq · mL⁻¹) | 2.6 | 664 | 681 | 457 | 373 |
| f₂ (%) | 0.2 | 3.1 | 3.4 | 2.2 | 1.8 |

LOQ = 1.4 ng-eq/mL

TABLE 10B
Radioactive concentration (ng-eq/mL) in blood after an
intravenous bolus of 1 mg/kg [¹⁴C]-42

| TIME | ANIMAL NO. | | | | |
|---|---|---|---|---|---|
| (h) | 01 | 02 | 03 | MEAN | SD |
| 0 | 0 | 1 | 1 | 1 | 1 |
| 0.08 | 722 | 772 | 655 | 716 | 58 |
| 0.5 | 305 | 232 | 306 | 281 | 42 |
| 1 | 131 | 146 | 99 | 125 | 24 |
| 2 | 63 | 66 | 55 | 61 | 6 |
| 4 | 34 | 38 | 48 | 40 | 7 |
| 8 | 18 | 26 | 32 | 26 | 7 |
| 32 | 11 | 13 | 15 | 13 | 2 |
| 48 | 6 | 15 | 15 | 12 | 5 |
| AUC (ng-eq · mL⁻¹) | 1524 | 2177 | 1987 | 1896 | 336 |

LOQ = 1.4 ng-eq/mL

TABLE 11A
Concentration (ng/mL) in blood after oral administration of
10 mg/kg [¹⁴C]-42

| TIME | ANIMAL NO. | | | | |
|---|---|---|---|---|---|
| (h) | 01 | 02 | 03 | MEAN | SD |
| 0.5 | 14.8 | 15.5 | 21.0 | 17.1 | 3.4 |
| 1 | 5.6 | 68.4 | 13.8 | 29.3 | 34.1 |
| 2 | 11.9 | 8.8 | 41.0 | 20.6 | 17.8 |
| 4 | 1.5 | 6.9 | 7.0 | 5.1 | 3.2 |
| 8 | 0.5 | 3.8 | 3.4 | 2.6 | 1.8 |
| 24 | 0.2 | 2.4 | 4.4 | 2.3 | 2.1 |
| 32 | 0.1 | 2.0 | 2.6 | 1.6 | 1.3 |
| 48 | 0.1 | 1.0 | 1.4 | 0.8 | 0.7 |

LOQ = 0.3 ng/mL

TABLE 11B
Concentration (ng/mL) in blood after intravenous bolus of
1 mg/kg [¹⁴C]-42

| TIME | ANIMAL NO. | | | | |
|---|---|---|---|---|---|
| (h) | 01 | 02 | 03 | MEAN | SD |
| 0.08 | 583.1 | 708.0 | 706.2 | 665.8 | 71.6 |
| 0.5 | 310.5 | 262.8 | 328.0 | 300.4 | 33.8 |
| 1 | 142.0 | 145.9 | 154.4 | 147.4 | 6.3 |
| 2 | 57.3 | 60.6 | 54.6 | 57.5 | 3.0 |
| 4 | 20.7 | 27.6 | 30.9 | 26.4 | 5.2 |
| 8 | 11.7 | 19.1 | 21.0 | 17.3 | 4.9 |
| 24 | 7.5 | ns | 10.4 | 9.0 | 2.1 |
| 32 | 7.2 | 13.6 | 9.1 | 10.0 | 3.3 |
| 48 | 5.8 | 7.3 | 5.7 | 6.2 | 0.9 |

LOQ = 0.3 ng/mL; ns = no sample

TABLE 12A
Pharmacokinetic parameters of 42 based on blood levels after oral
administration of 10 mg/kg [¹⁴C]-42

| | | ANIMAL NO. | | | | |
|---|---|---|---|---|---|---|
| PARAMETERS | UNIT | 01 | 02 | 03 | MEAN | SD |
| Cₘₐₓ | (ng/mL) | 14.8 | 68.4 | 41.0 | 41.4 | 26.8 |
| Tₘₐₓ | (h) | 0.5 | 1.0 | 2.0 | 1.2 | 0.8 |
| t_{½} | (h) | 2 | 16 | 22 | 13 | 10 |
| AUC | (ng-mL⁻¹-h) | 39 | 217 | 280 | 179 | 125 |
| f | (%) | 0.3 | 1.5 | 2.2 | 1.3 | 0.9 |

TABLE 12B
Pharmacokinetic parameters based on blood levels after intravenous bolus of 1
mg/kg [¹⁴C]-42

| | | ANIMAL NO. | | | | |
|---|---|---|---|---|---|---|
| PARAMETERS | UNIT | 01 | 02 | 03 | MEAN | SD |
| AUC | (ng-mL⁻¹-h) | 1167 | 1490 | 1282 | 1313 | 164 |
| AUMC | (ng-mL⁻¹-h²) | 31393 | 37347 | 20234 | 29658 | 8688 |
| T_{1/221} | (h) | 0.53 | 0.44 | 0.37 | 0.45 | 0.08 |
| t_{½} | (h) | 30 | 26 | 15 | 24 | 7 |
| f₂ | (%) | 61 | 72 | 66 | 67 | 6 |
| MRT | (h) | 27 | 25 | 16 | 23 | 6 |
| CL | (mL/min) | 5.1 | 3.8 | 4.5 | 4.5 | 0.7 |
| Vss | (1/kg) | 26 | 19 | 14 | 20 | 6 |

TABLE 13
Radioactivity concentrations (ng-eq/g) in brain and blood at 0.5 hours
after an intravenous blood of 1 mg/kg [¹⁴C]-42

| | INDIVIDUAL VALUES | | | MEAN ± SD |
|---|---|---|---|---|
| Brain | 6 | 8 | 6 | 7 ± 1 |
| Blood | 236 | 265 | 184 | 228 ± 41 |
| Kp (Brain/Blood) | 0.03 | 0.03 | 0.03 | 0.03 ± .003 |

TABLE 14
Brain extraction ratios (%) of [¹⁴C]-42 obtained 5 s after intracarotid injection of
the test compound dissolved either in Ringer's buffer (n = 6 rats) or in blank rat
plasma (n = 5 rats)

| INJECTION VEHICLE | INDIVIDUAL VALUES | | | | | | MEAN ± SD |
|---|---|---|---|---|---|---|---|
| Ringer's buffer | 27.3 | 49.8 | 45.3 | 34.9 | 38.5 | 50.3 | 41.0 ± 9.1 |
| Rat plasma | 2.8 | 3.0 | 1.8 | 3.0 | 2.6 | — | 2.7 ± 0.5 |

TABLE 15
Concentration and temperature dependence for the blood distribution
of [¹⁴C]-42 in rats

| | BLOOD CONCENTRATION (NG/ML) | | | |
|---|---|---|---|---|
| TEMPERATURE | 5 | 50 | 500 | 5000 |
| 4° C. | 91.6 ± 5.5 | 88.4 ± 1.8 | 84.9 ± 3.5 | 68.9 ± 1.8 |
| 22° C. | 66.8 ± 1.9 | 57.4 ± 1.0 | 53.7 ± 0.4 | 62.6 ± 1.5 |
| 37° C. | 59.9 ± 0.3 | 41.8 ± 0.8 | 37.8 ± 0.7 | 40.4 ± 0.3 |

Results represent the percentage of total drug mass in plasma (mean ± SD of triplicate determination)

TABLE 16
Fraction free of [¹⁴C]-42 in plasma of rats and human at 37° C.

| | PLASMA CONCENTRATION (NG/ML) | | |
|---|---|---|---|
| SPECIES | 50 | 500 | 5000 |
| Rat | 10 ± 2 | 11 ± 0.3 | 12 ± 1 |
| Human | 15 ± 1 | 23 ± 2 | 21 ± 1 |

Results represent free fractions (%) in plasma (mean ± SD of triplicate determination)

TABLE 17
Urinary excretion (% of dose) of radioactivity and parent drug
during 0-48 hours after a singe oral (10 mg/kg) or intravenous
(1 mg/kg) does of [¹⁴C]-42

| | | ANIMAL NO. | | | | |
|---|---|---|---|---|---|---|
| | | 1 | 2 | 3 | MEAN | SD |
| p.o. | Radioactivity | 0.03 | 0.13 | 0.25 | 0.14 | 0.11 |
| | 42 | 0.001 | 0.053 | 0.114 | 0.056 | 0.057 |
| i.v. | Radioactivity | 1.3 | 3.3 | 3.1 | 2.5 | 1.1 |
| | 42 | 0.96 | 1.98 | 1.97 | 1.64 | 0.58 |

The numbers represent underestimates because some urine was lost during blood sampling.

It should be understood that the examples and embodiments described herein are for illustrative purposes only and that various modifications or changes in light thereof will be suggested to persons skilled in the art and are to be included within the spirit and purview of this application and the scope of the appended claims.

We claim:

1. A compound of formula: 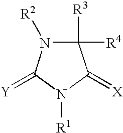
wherein R¹ is —(CH₂)₅—NH₂; R² is —SO₂-3-(CN)-4-(p-NH₂-Ph-O)-Ph; R³ is H; and R⁴ is R3-CH₂-3-indolyl; X and Y are O.

2. A compound of formula: 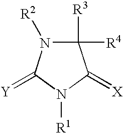
wherein R¹ is —(CH₂)₅—NH₂,; R² is —SO₂-3-[(Me)₂NCO]-4-(3-Cl-4-OH-Ph-O)-Ph; R³ is H; and R⁴ is —CH₂-3-(7-Me-indolyl); X and Y are O.

3. A compound of formula: 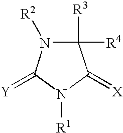
wherein R¹ is —(CH₂)₅—NH₂,; R² is —SO₂-3-(CN)-4-(3-Cl-4-OH-Ph-O)-Ph; R³ is H; and R⁴ is —CH₂-3-(7-Me-indolyl); X and Y are O.

4. A compound of formula: 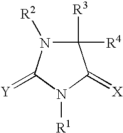
wherein R¹ is —(CH₂)₅—NH₂,; R² is —SO₂-3-(CH₂NH₂)-4-(p-OH-Ph-O)-Ph; R³ is H; and R⁴ is —CH₂-3-(7-Me-indolyl); X and Y are O.

5. A compound of formula: 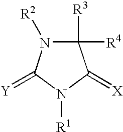
wherein R¹ is —(CH₂)₅—NH₂, R² is —SO₂-3-[(Me)₂NCO]-4-(p-OH-Ph-O)-Ph-; R³ is H; and R⁴ is —CH₂-3-(7-Me-indolyl); X and Y are O.

6. A compound of formula: 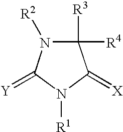
wherein R¹ is —(CH₂)₄—C(CH₃)₂—NH₂, R² is —SO₂-3-[(Me)₂NCO]-4-(3-Cl-4-OH-Ph-O)-Ph; R³ is H; and R⁴ is —CH₂-3-(7-Me-indolyl); X and Y are O.

7. A compound of formula: 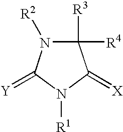
wherein R¹ is —(CH₂)₅—NH₂,; R² is —SO₂-3-[(Me)₂NCO]-4-[3-Cl-4-(OCOCH(NH₂)(CH₂)₄NH₂)—Ph-O]-Ph; R³ is H; and R⁴ is —CH₂-3-indolyl; X and Y are O.
